(19) United States
(12) Patent Application Publication
(10) Pub. No.: US 2026/0301893 A1
(43) Pub. Date: Oct. 1, 2026

(54) SELECTION OF A FITTING WINDOW FOR TRANSDERMAL GLOMERULAR FILTRATION RATE, DETECTION OF SENSOR DISPLACEMENT, AND END OF SESSION DETECTION

(71) Applicant: MediBeacon Inc., St. Louis, MO (US)

(72) Inventors: Martin Debreczeny, Berkley, CA (US); Augustus Lowell, Durham, NH (US); Jesse Ross-Jones, Wittlich (DE); Jochen Friedemann, Altrip (DE)

(21) Appl. No.: 19/575,384

(22) Filed: Mar. 23, 2026

Related U.S. Application Data
(60) Provisional application No. 63/778,200, filed on Mar. 26, 2025.
(60) Provisional application No. 63/911,973, filed on Nov. 5, 2025.

(51) Int. Cl. G16H 10/40; G16H 50/30

(52) U.S. Cl. CPC G16H 10/40; G16H 50/30

(57) ABSTRACT

Method, system, and apparatus of transdermal glomerular filtration rate (tGFR) fitting window selection includes obtaining a measurement data set including a plurality of measurement entries obtained before or after administration of an exogenous fluorescent agent. A renal decay time constant (RDTC) is calculated for a predetermined window having a window length greater than or equal to a minimum window length and equal to or smaller than a maximum window length. A quality factor (QF) corresponding to the predetermined window is computed, the QF including a signal-to-noise component and a signal level component. A tGFR is calculated using data spanning the predetermined window, and the calculated tGFR is reported.

### TECHNICAL FIELD

The present disclosure relates generally to methods and systems for monitoring phases of an exogenous fluorescence agent administration session and evaluation thereof.

### BACKGROUND

Monitoring of a biological parameter in patients that are critically ill or injured is important. Organ function can be impaired due to organ damage, aging, an underlying illness, and others. Assessing organ function allows for determination of an organ damage and/or organ failure.

### BRIEF DESCRIPTION OF THE DRAWINGS

Details of one or more aspects of the subject matter described in this disclosure are set forth in the accompanying drawings and the description below. However, the accompanying drawings illustrate only some typical aspects of this disclosure and are therefore not to be considered limiting of its scope. Other features, aspects, and advantages will become apparent from the description, the drawings and the claims.

In order to describe the manner in which the above-recited and other advantages and features of the disclosure can be obtained, a more particular description of the principles briefly described above will be rendered by reference to specific examples thereof which are illustrated in the appended drawings. Understanding that these drawings depict only exemplary examples of the disclosure and are not, therefore, to be considered to be limiting of its scope, the principles herein are described and explained with additional specificity and detail through the use of the accompanying drawings in which:

FIG. 1 is a schematic illustration of a single light source monitoring device in one aspect;

FIG. 2 illustrates illumination from a light source and exogenous fluorescence agent, and detection by a first detector and a second detector;

FIG. 3 illustrates illumination of light from another light source and detection by a first detector and a second detector;

FIG. 4 is a schematic illustration of a multi-light source monitoring system in one aspect;

FIG. 5 illustrates an example of a sensor head having one or more sources and two detectors;

FIG. 6 is an exploded view of the inner housing of the sensor head illustrated in FIG. 5;

FIG. 7 illustrates an example graph of a fluorescence signal before and after administration of an exogenous fluorescent agent;

FIG. 8 illustrates an example flow-chart of multi-window GFR estimation algorithm;

FIGS. 9A-9B illustrates example graphs of QF_{SNR} vs. a fitting window, and QFMₗᵥₗ vs. IF;

FIG. 10 illustrates an example diagram of a method for transdermal Glomerular Filtration Rate (tGFR) fitting window selection;

FIG. 11 illustrates a method for predicting a time until an End of a GFR Session;

FIG. 12 illustrates a method for sensor displacement detection;

FIG. 13 illustrates an example flow-chart of computing or calculating IF signals;

FIG. 14 illustrates an example flow-chart of End of Session detection;

FIG. 15 illustrates a flow-chart corresponding to multi-window GFR estimation using an exogenous fluorescent agent in a patient.

### DETAILED DESCRIPTION

Various examples of the disclosure are discussed in detail below. While specific implementations are discussed, it should be understood that this is done for illustration purposes only. A person skilled in the relevant art will recognize that other components and configurations can be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an example in the present disclosure can be references to the same example or any example; and such references mean at least one of the examples.

Reference to “one example” or “an example” means that a particular feature, structure, or characteristic described in connection with the example is included in at least one example of the disclosure. The appearances of the phrase “in one example” in various places in the specification are not necessarily all referring to the same example, nor are separate or alternative examples mutually exclusive of other examples. Moreover, various features are described which can be exhibited by some examples and not by others.

The terms used in this specification generally have their ordinary meanings in the art, within the context of the disclosure, and in the specific context where each term is used. Alternative language and synonyms can be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various examples given in this specification.

Without intent to limit the scope of the disclosure, examples of instruments, apparatus, methods and their related results according to the examples of the present disclosure are given below. Note that titles or subtitles can be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions, will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein. Additionally, unless specifically required the order of one or more of the steps can be as described or the order can be adapted.

As used herein, the terms “comprises,” “comprising,” “includes,” “including,” “has,” “having” or any other variation thereof, are intended to cover a non-exclusive inclusion. For example, a process, product, article, or apparatus that comprises a list of elements is not necessarily limited only those elements but can include other elements not expressly listed or inherent to such process, article, or apparatus. Further, unless expressly stated to the contrary, “or” refers to an inclusive or and not to an exclusive or. For example, a condition A or B is satisfied by any one of the following: A is true (or present), and B is false (or not present), A is false (or not present), and B is true (or present), and both A and B are true (or present).

The term substantially, as used herein, is defined to be essentially conforming to the particular dimension, shape or other word that substantially modifies, such that the component need not be exact. For example, substantially cylindrical means that the object resembles a cylinder, but can have one or more deviations from a true cylinder.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series and the like.

The phrase “diffuse reflecting medium” refers to any material through which light propagates, which includes a plurality of moieties, particles, or molecules that can scatter, reflect, and/or absorb the light as it propagates. The distribution of the plurality of moieties, particles, and/or molecules can be uniform or non-uniform and can change over time. A diffuse reflectance (DR) signal refers to light scattered and reflected off a diffuse reflecting medium, such as, but not limited to, organ tissue, skin tissue, etc., or other inanimate diffuse reflecting media.

To understand a patient's renal health, it is important to measure glomerular filtration rate (GFR), which indicates how effectively the kidneys are filtering waste and excess fluid from the bloodstream. Traditional methods for determining GFR relied on invasive techniques involving the collection of urine or blood samples. Urine-based methods required extended collection periods to measure the clearance of specific substances, while blood-based methods estimated GFR indirectly using biomarkers such as creatinine or cystatin C. These approaches were often time-consuming, prone to errors, and lacked the ability to provide real-time or continuous monitoring of kidney function, limiting their utility in critical care settings.

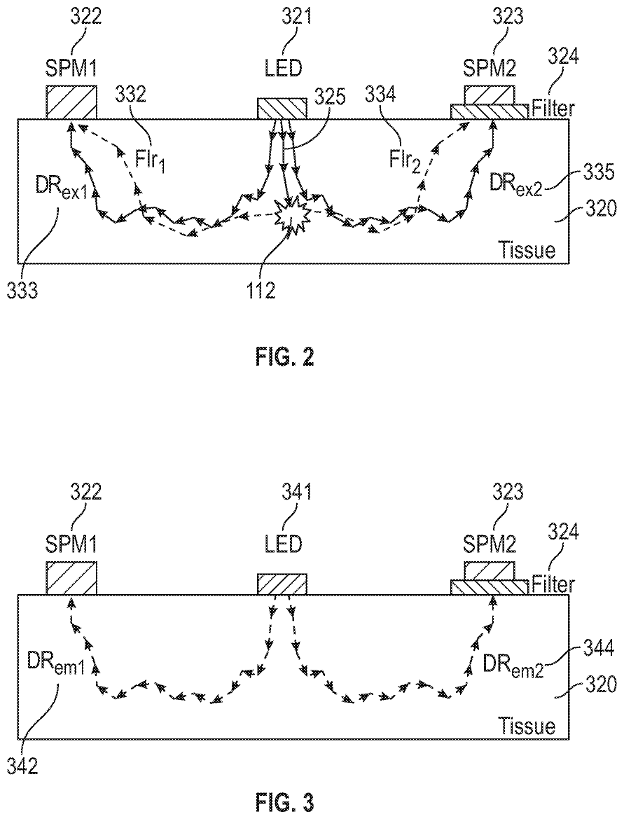

A more precise but less invasive method for measuring GFR is accomplished by administering to a patient an exogenous fluorescent agent to enable non-invasive monitoring of organ function, particularly kidney function, through the measurement of transdermal Glomerular Filtration Rate (tGFR). The tGFR reflects how efficiently the kidneys are filtering the agent from the bloodstream, which is a critical indicator of renal health. The exogenous fluorescent agent may be administered to a patient during a diagnostic session for monitoring the patient's kidney function. One or more sensors, such as those described below (see, e.g., FIGS. 2-6), are used to measure the IF of the exogenous fluorescent agent as the exogenous fluorescent agent is processed by the kidneys in accordance with the tGFR.

In some aspects, the techniques described herein relate to a method of transdermal Glomerular Filtration Rate (tGFR) fitting window selection including: obtaining a measurement data set including a plurality of measurement entries, wherein each measurement entry of the plurality of measurement entries is obtained before or after administration of an exogenous fluorescent agent; calculating a renal decay time constant (RDTC), wherein the RDTC corresponds to a predetermined window that has a window length that is equal to or greater than a minimum window length and equal to or smaller than a maximum window length; computing a quality factor (QF) corresponding to the predetermined window, wherein the QF includes a QF_{SNR} component and a QF_{LVL} component; calculating a tGFR for the predetermined window, wherein the tGFR is calculated using data spanning the predetermined window; and reporting the calculated tGFR.

In some aspects, the techniques described herein relate to a method of detecting sensor displacement for a system operable to measure Glomerular Filtration Rate using an exogenous fluorescent agent, the method including: generating an intrinsic fluorescence (IF) signal representing a detected fluorescence intensity emitted by the exogenous fluorescent agent from within a diffuse reflecting medium based on measurements from one or more sensors; detecting a change in the IF signal corresponding to potential displacement of the one or more sensors relative to a patient, wherein the potential displacement corresponds with a perturbation in at least one raw optical signal; determining a peak has been reached in the IF signal; continuing to calculate the IF signal for one or more predetermined time periods if the peak has been reached and comparing IF signal after each of the one or more predetermined time periods relative to a predicted IF signal based on a calculation from a selected time prior to the potential displacement; continuing with measurements when the calculated IF signal is less than or equal to a predetermined deviation from the expected IF value; and declaring an End of Session if the calculated IF signal is more than the predetermined deviation from the expected IF value.

In some aspects, the techniques described herein relate to a method of detecting an end of a Glomerular Filtration Rate (GFR) session using an exogenous fluorescent agent, the method including: computing a quality factor (QF) corresponding to a predetermined window of measurement data set including a plurality of measurement entries obtained before or after administration of an exogenous fluorescent agent, wherein the QF includes a QF SNR component and a QF_{LVL} component; calculating a transdermal Glomerular Filtration Rate (tGFR) for the predetermined window, wherein the tGFR is calculated using data spanning the predetermined window; assigning a lower of QF_{SNR} or QF_{LVL} as an end determining QF; and declaring an End of Session once the end determining QF falls below a limiting tolerance level.

In some aspects, the techniques described herein relate to a method of predicting a time until an end of a Glomerular Filtration Rate (GFR) session using an exogenous fluorescent agent, the method including: obtaining a measurement data set including a plurality of measurement entries that are obtained before and after administration of an exogenous fluorescent agent; generating an intrinsic fluorescence (IF) signal representing a detected fluorescence intensity emitted by the exogenous fluorescent agent from within a diffuse reflecting medium; saving a last known good signal level as IF_{good}; saving a last known good RDTC value as RDTC_{good}; calculating the time until the end of the GFR session based upon when an End of Session time will occur such that an End of Session will be declared, wherein the End of Session time is a function of a current signal level and a current signal to noise ratio.

The present disclosure provides methods of signal evaluation for monitoring exogenous fluorescent agent by analyzing an intrinsic fluorescence (IF) signal emitted by the exogenous fluorescent agent as it is processed by the body.

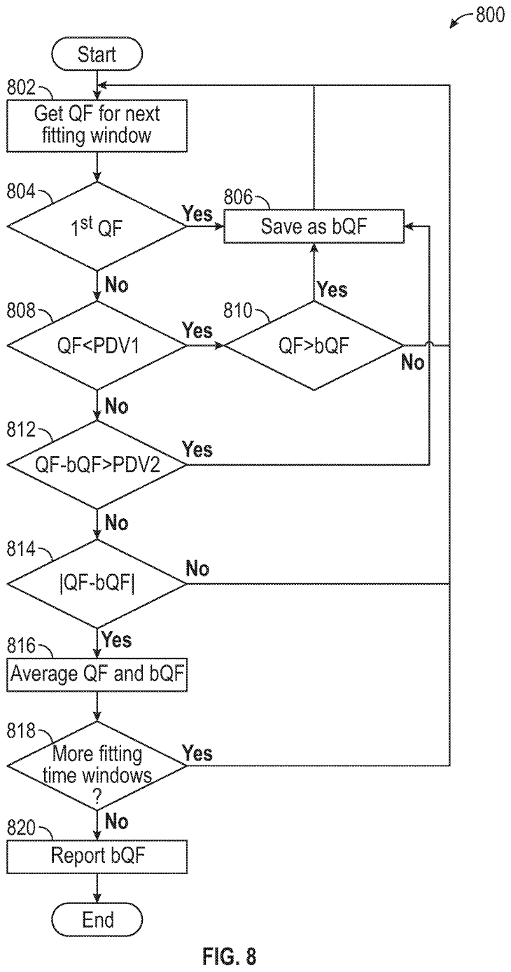

This disclosure provides a process for selecting fitting windows for tGFR calculation by computing a renal decay time constant (RDTC) and assessing data quality through a Quality Factor (QF) that incorporates signal-to-noise and signal level components. A fitting window can be used for calculating the transdermal Glomerular Filtration Rate (tGFR). The renal decay time constant (RDTC) may be calculated, wherein the RDTC corresponds to a predetermined window that has a window length that is equal to or greater than a minimum window length. A particular fitting window size is selected in accordance with data availability. For example, at early times, the window size may be limited; however, the available window sizes may expand with time. For each fitting window size, a quality factor (QF) is computed. The QF may be computed for each fitting window, wherein the QF includes a QF_{SNR} component and a QFₗᵥₗ component. As described in further detail with respect to FIG. 8, the fitting window size that produces a QF greater than a predetermined threshold is chosen, or several windows that have roughly the same QF are averaged. The QF ensures that the measurement data spanning a given fitting window is sufficient to provide a valid determination of the RDTC of the fitting window, and thus, the tGFR determined using the RDTC.

The present disclosure also provides non-invasive monitoring of a biological parameter indicative of organ function in a patient based upon an IF signal. The present disclosure makes use of a unique filtering of the IF signal to determine if the IF signal includes a portion of premature fluorescence data and identifies, upon determination of the portion of premature fluorescence data, an operable IF signal range by filtering the IF signal associated with premature fluorescence data. The IF signal is based upon use of a suitable indicator that is administered to a patient. In one example, the administration to the patient can be through injection. In another example, the administration to the patient can be through ingestion. In one example, the organ function is intestinal wall barrier function wherein the intestinal wall barrier function in a patient is assessed based upon an IF signal. In at least one example, the organ function is renal function. The present disclosure increases accuracy of calculating the renal function in a patient based upon an IF signal.

Suitable indicator substances for use with the methods and devices described herein are disclosed in U.S. Pat. Nos. 8,155,000, 8,664,392, 8,697,033, 8,703,100, 8,722,685, 8,778,309, 9,005,581, 9,283,288, 9,376,399, RE47,413, RE47,255, 10,137,207, 10,525,149, and 11,590,244, which are all incorporated by reference in their entirety for all purposes. In some aspects, the indicator substance is eliminated from the body of a patient by glomerular filtration. In some aspects, the indicator substance is eliminated from the body of a patient only by glomerular filtration. In some aspects, the indicator substance is a GFR agent.

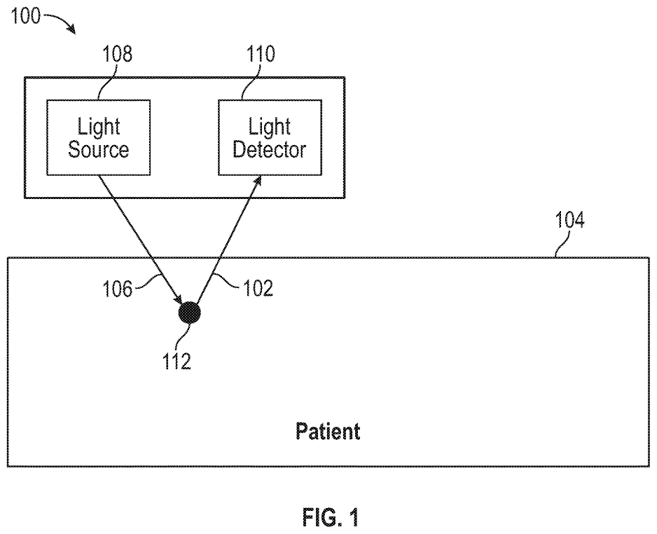

FIG. 1 is a schematic illustration of a system 100 in which an exogenous fluorescent agent 112 is administered to a patient. A light source 108 emits light 106 into the patient 104. The light 106 can be described as an excitation light or an excitation light source. The light 106 can also be controlled to be at one wavelength, multiple wavelengths that vary over time, or multiple wavelengths emitted simultaneously. The exogenous fluorescent agent 112 produces fluorescence 102 in response to an excitation event including: illumination by light 106 at an excitation wavelength (λₑₓ), occurrence of an enzymatic reaction, changes in local electrical potential, and any other known excitation event associated with exogenous fluorescent agents. The light source 108 can be configured to deliver light 106 at an excitation wavelength (λₑₓ) to the patient 104. Fluorescence 102 includes an emission wavelength produced as the emitted light 106 is absorbed by and then reflected from the patient 104. In at least one example, the excitation wavelength (λₑₓ) of the light 106 and the emission wavelength (λₑₘ) of the fluorescence 102 can be spectrally distinct ((λₑₓ) is sufficiently different from (λₑₘ) so that the light detector 110 can be configured to selectively detect only the fluorescence 102 by the inclusion of any known optical wavelength separation device including an optical filter).

Change in the fluorescence 102 can be analyzed to obtain information regarding organ function of the patient 104. As described herein, two non-limiting examples of organ function can be one of renal function and/or intestinal wall barrier function. In one example, the rate of decrease in fluorescence 102 can be proportional to the rate of removal of the exogenous fluorescent agent 112 by one or more organs of the patient 104, thereby providing a biological parameter value. In another non-limiting example, the rate of decrease in fluorescence 102 can be proportional to the rate of removal of the exogenous fluorescent agent 112 by the kidneys of the patient 104, thereby providing a measurement of renal function including: renal decay time constant (RDTC) and/or glomerular filtration rate (GFR).

FIG. 2 illustrates using a light source 108 in the form of an excitation light emitting diode (LED) 321 in the presence of the exogenous fluorescent agent 112. The excitation LED 321 can emit light at one or more different excitation wavelengths. In one example, the excitation light wavelength can be a blue light. In other examples, the excitation light wavelength can be a blue light and a green light. In other examples, the excitation light wavelength can be chosen based upon the selected exogenous fluorescent agent 112. The light detector 110 can be in the form of a first light detector 322 and a second light detector 323. As illustrated the first light detector 322 can receive a first signal labeled SPM1 and the second light detector 323 can receive a second signal labeled SPM2. In at least one example, the first light detector 322 and the second light detector 323 can each be a silicon photomultiplier (SPM). While the light detectors 322, 323 can take a variety of different forms, the silicon photomultiplier and/or photo diodes can provide for desired characteristics to perform the measurements to achieve the desired accuracy for these measurements.

Additionally, a filter 324 can be configured to filter out light prior to the second detector 323 receiving the light. The filter 324 can be configured to substantially or fully block excitation light wavelength. Additionally, the filter 324 can be configured to allow light that is emitted from the exogenous fluorescent agent to pass therethrough substantially unimpeded. In the illustrated example, the excitation light wavelength can be a blue wavelength and the filter 324 can be configured to allow green light to pass therethrough. As a result, the first detector 322 is configured to measure light received at both the excitation and emission wavelengths, and the second detector 323 is configured to detect light received at the emission wavelength only. Combined with the illumination of the tissues 320 of the patient 104 with light at the excitatory wavelength only and at the emission wavelength only in an alternating series, the measurements from the first detector 322 and a second detector 323 may be analyzed as described in U.S. Pat. Nos. 10,548,521, 10,980,459, 10,952,656, 11,478,172 and 10,194,854 to measure the fluorescence of an exogenous fluorescence agent and to correct the fluorescence measurements by removing the effects of autofluorescence, excitation-wavelength light leak-through and the diffuse reflectance of light according to the correction methods described therein. While the illustrated example only includes a single filter 324, in examples an additional filter can be configured to filter out light prior to the first light detector 322. In other examples, a single filter can be placed before the first light detector 322 rather than the second light detector 323.

The excitation LED 321 (for example, a blue LED) can emit light 325 that is directed toward the exogenous fluorescent agent 112. Additionally, light emitted from the excitation LED 321 can travel through the patient 104 such that the tissue 320 of the patient serves to diffuse the light. The diffused light can be referred to as a diffuse reflectance (DR) signal. Additionally, the light 325 that impacts the exogenous fluorescent agent 112 and the fluorescence emission (Flr) signal 334 travel to the detectors 322, 323. As illustrated, the first detector 322 receives a DR signal 333 labeled as DRex₁ and a Flr signal 332 labeled as Flr₁, and the second detector 323 receives a DR signal 335 labeled as DRex₂ and a Flr signal 334 labeled as Flr₂. The Flr signals 332 and 334 include contributions from the exogenous fluorescent agent 112 and tissue autofluorescence. These measurements are used to arrive at intrinsic fluorescence (IF) signal that is just of the agent as described herein.

FIG. 3 illustrates using a light source 108 in the form of another light emitting diode (LED) 341. In one example, the another LED 341 can be a green LED. In yet other examples, the another LED 341 can be other types of LEDs that provide light at a different wavelength from the excitation LED 321. In one example, the another LED 341 can be operable to emit light in substantially the same wavelength as the emission from the exogenous agent. In other examples, a single LED capable of emitting light at various wavelengths can be implemented. Light emitted from the another LED 341 can travel through the patient 104 such that the tissue 320 of the patient serves to diffuse the light. The diffuse light can be referred to a DR signal, as described herein. As illustrated, the first detector 322 receives a DR signal 342 labeled as DRem₁, and the second detector 323 receives a DR signal 344 labeled as DRem₂. As in FIG. 2, a filter 324 can be implemented. This filter can be the same as in FIG. 2. While the another LED 341 and excitation LED 321 are indicated as being separate from one another, the another LED 341 and excitation LED 321 can be coupled to one another. While the illustrated example only includes a single filter 324, in examples an additional filter can be configured to filter out light prior to the first detector 322. In other examples, a single filter can be placed before the first detector 322 rather than the second detector 323.

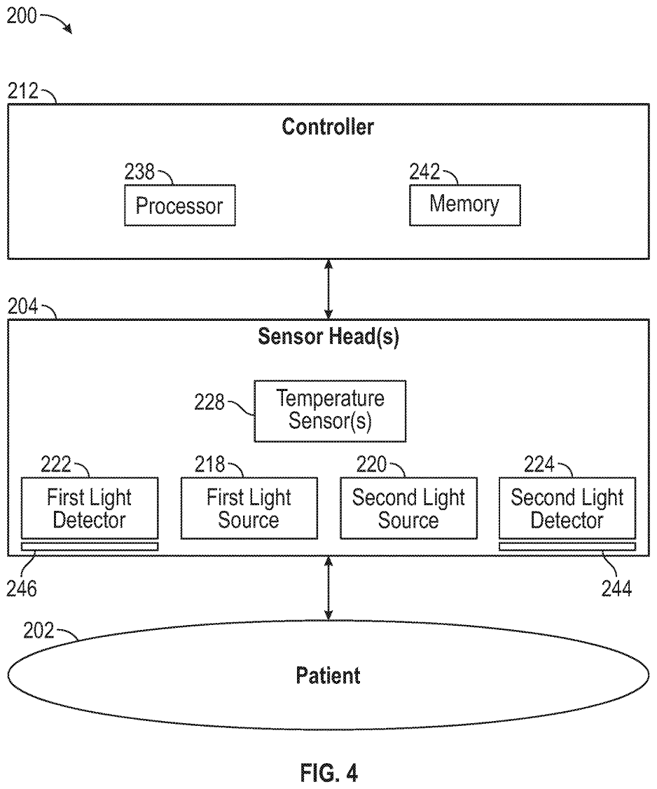

FIG. 4 is an example schematic illustration of an organ monitoring system 200. The system can include a controller 212 that includes a processor 238 and a memory 242. The controller 212 can be coupled to one or more sensor heads 204. Each sensor head can include a first light source 218 and a second light source 220. The first light source 218 can be an excitation LED as indicated above. The second light source 220 can be another LED as indicated above. In other examples, the excitation LED can be the second light source 220 and the another LED can be the first light source 218. As illustrated a first light filter 246 and a second light filter 244 is included. In other examples, only one of the first light filter 246 and second light filter 244 can be implemented, such as described in regards to FIG. 2 and FIG. 3. The sensor head 204 can optionally include one or more temperature sensor(s) 228. The one or more temperature sensor(s) 228 can collect data at the same time as the data being collected from the first light detector 222 and/or second light detector 224. The one or more temperature sensor(s) 228 can be used to determine characteristics associated with the first light detector 222 and/or second light detector 224. Additionally, the one or more temperature sensor(s) 228 can be arranged to provide information regarding the patient 202. While the illustrated example includes the controller 212 as separate from the sensor head(s) 204, in other examples the controller 212 and sensor head 204 can be part of a single unit rather than being coupled either wired or wirelessly.

The first light source 218 and second light source 220 can be configured to emit light into the patient 202. The light can be diffused within the patient and a portion of the light is received at the first light detector 222 and/or a portion of the light is received at the second light detector 224. The data obtained by the first light detector 222 and/or the second light detector 224 can be transmitted to the controller 212. The data can be stored in memory 242 or another storage device with which the controller is in electronic communication. In examples, the data obtained includes measurement data comprising a plurality of measurement entries, wherein each measurement entry of the plurality of measurement entries is obtained before or after administration of the exogenous fluorescent agent.

The processor 238 can be operable to execute instructions according to one or more methods as described herein. The processor 238 can be operable to calculate a biological parameter value. In one example, the biological parameter value can be one or more of a GFR and/or RDTC. In other examples, the biological parameter value can be a parameter to describe permeability and/or leaks of the intestinal wall.

In various aspects, the first light source 218 and the second light source 220 can be any light source configured to deliver light at the excitatory wavelength and at the emission wavelength. Typically, the first light source 218 delivers light at an intensity that is sufficient to penetrate the tissues of the patient 202 to the exogenous fluorescent agent with sufficient intensity remaining to induce light at the emission wavelength by the exogenous fluorescent agent. Typically, the first light source 218 delivers light at an intensity that is sufficient to penetrate the tissues of the patient 202 to the exogenous fluorescent agent with sufficient intensity remaining after scattering and/or absorption to induce fluorescence at the emission wavelength by the exogenous fluorescent agent. However, the intensity of light delivered by the first light source 218 is limited to an upper value to prevent adverse effects such as tissue burning, tissue tanning, cell damage, and/or photo-bleaching of the exogenous fluorescent agent and/or the endogenous chromophores in the skin (“auto-fluorescence”).

Similarly, the second light source 220 delivers light at the emission wavelength of the exogenous fluorescent agent at an intensity configured to provide sufficient energy to propagate with scattering and absorption through the first region of the patient and out the second region and third region with sufficient remaining intensity for detection by the first light detector 222 and the second light detector 224, respectively. As with the first light source 218, the intensity of light produced by the second light source 220 is limited to an upper value to prevent the adverse effects such as tissue burning, tissue tanning, cell damage, and/or photo-bleaching of the exogenous fluorescent agent and/or the endogenous chromophores in the skin (“auto-fluorescence”).

In various aspects, the first light source 218 and the second light source 220 can be any light source suitable for use with fluorescent medical imaging systems and devices. Non-limiting examples of suitable light sources include: LEDs, diode lasers, pulsed lasers, continuous wave lasers, xenon arc lamps or mercury-vapor lamps with an excitation filter, lasers, and supercontinuum sources. In one aspect, the first light source 218 and/or the second light source 220 can produce light at a narrow spectral bandwidth suitable for monitoring the concentration of the exogenous fluorescence agent using the methods described herein. In another aspect, the first light source 218 and the second light source 220 can produce light at a relatively wide spectral bandwidth.

In one aspect, the selection of intensity of the light produced by the first light source 218 and the second light source 220 by the system 200 can be influenced by any one or more of at least several factors including, but not limited to, the maximum permissible exposure (MPE) for skin exposure to a laser beam according to applicable regulatory standards such as ANSI standard Z136.1. In another aspect, light intensity for the system 200 can be selected to reduce the likelihood of photobleaching of the exogenous fluorescent source and/or other chromophores within the tissues of the patient 202 including, but not limited to: collagen, keratin, elastin, hemoglobin within red blood cells and/or melanin within melanocytes. In yet another aspect, the light intensity for the system 200 can be selected in order to elicit a detectable fluorescence signal from the exogenous fluorescent source within the tissues of the patient 202 and the first light detector 222 and/or second light detector. In yet another aspect, the light intensity for the system 200 can be selected to provide suitably high light energy while reducing power consumption, inhibiting heating/overheating of the first light source 218 and the second light source 220, and/or reducing the exposure time of the patient's skin to light from the first light detector 222 and/or second light detector.

In various aspects, the intensity of the first light source 218 and the second light source 220 can be modulated to compensate any one or more of at least several factors including, but not limited to: individual differences in the concentration of chromophores within the patient 202, such as variation in skin pigmentation. In various other aspects, the detection gain of the light detectors can be modulated to similarly compensate for variation in individual differences in skin properties. In an aspect, the variation in skin pigmentation can be between two different individual patients 202, or between two different positions on the same patient 202. In an aspect, the light modulation can compensate for variation in the optical pathway taken by the light through the tissues of the patient 202. The optical pathway can vary due to any one or more of at least several factors including but not limited to: variation in separation distances between the light sources and light detectors of the system 200; variation in the secure attachment of the sensor head 204 to the skin of the patient 202; variation in the light output of the light sources due to the exposure of the light sources to environmental factors such as heat and moisture; variation in the sensitivity of the light detectors due to the exposure of the light detectors to environmental factors such as heat and moisture; modulation of the duration of illumination by the light sources, and any other relevant operational parameter.

In various aspects, the first light source 218 and the second light source 220 can be configured to modulate the intensity of the light produced as needed according to any one or more of the factors described herein above. In one aspect, if the first light source 218 and the second light source 220 are devices configured to continuously vary output fluence as needed, for example LED light sources, the intensity of the light can be modulated electronically using methods including, but not limited to, modulation of the electrical potential, current, and/or power supplied to the first light source 218 and/or the second light source 220. In another aspect, the intensity of the light can be modulated using optical methods including, but not limited to: partially or fully occluding the light leaving the first light source 218 and the second light source 220 using an optical device including, but not limited to: an iris, a shutter, and/or one or more filters; diverting the path of the light leaving the first light source 218 and the second light source 220 away from the first region of the patient using an optical device including, but not limited to a lenses, a mirror, and/or a prism.

In various aspects, the intensity of the light produced by the first light source 218 and the second light source 220 can be modulated via control of the laser fluence, defined herein as the rate of energy within the produced light beam. In one aspect, the laser fluence can be limited to ranges defined by safety standards including, but not limited to, ANSI standards for exposure to laser energy such as ANSI Z136.1.

In various aspects, the pulse width of the light produced by the first light source 218 and the second light source 220 can be independently selected to be a duration ranging from about 0.0001 seconds to about 0.5 seconds.

In various aspects, a measurement data set detected and/or sensed by the organ monitoring system 200 may include a variety of measurements taken at a preselected interval (e.g., every 50 milliseconds (ms), 100 ms, 500 ms, 1 second(s), 10 s, or greater). The measurement data may include SPM1 and/or SPM2, each demodulated, averaged, and/or peak thereof, during illumination with green and/or blue light, and/or with no illumination. The measurement data may include a photodiode signal demodulated, averaged, and/or a peak thereof during illumination with green light and/or blue light, and/or with no illumination. In aspects, the measurement data set may include bias voltage sensed during at least one illumination by green and/or blue light, or without illumination. The measurement data set may include any measurements of a fluorescence signal and/or other physical parameters that may be affected by the administration of the exogenous fluorescent agent or illumination by a light source (e.g., light source 108, light sources 218).

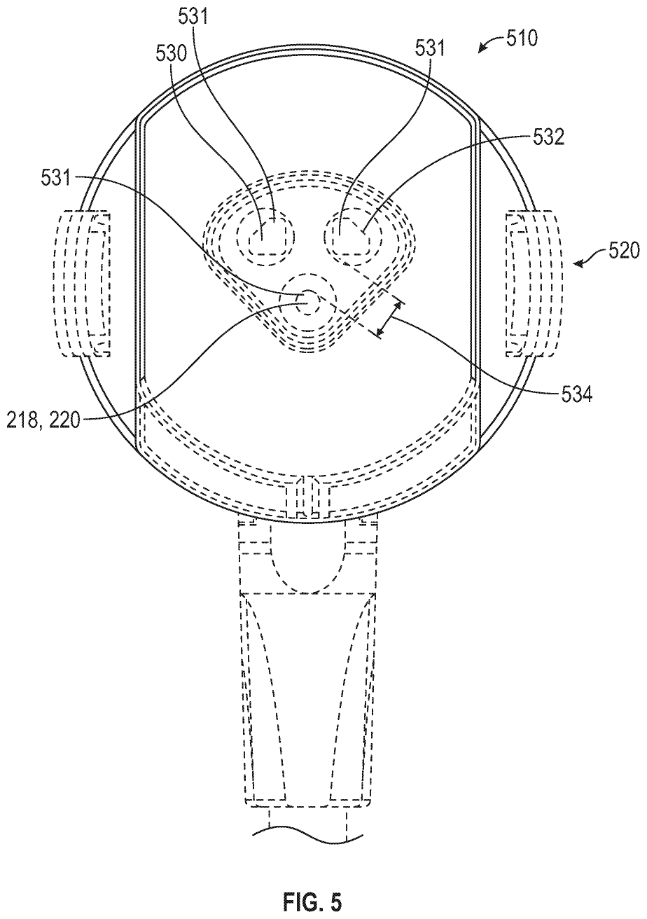

FIG. 5 illustrates an example of a sensor head having one or more light sources and two or more light detectors. As illustrated, a single aperture 531 formed in the sensor head 510 allows light from a first light source 218 and a second light source 220 to pass therethrough. The sensor head 510 also includes a first detector 530 and a second detector 532. Respective apertures 531 can be formed in the sensor head 510 to allow light to reach the first detector 530 and/or the second detector 532. A distance 534 separates the second detector 532 from the one or more light sources 218, 220. The sensor head 510 can also include clip receivers 520 that are designed to be coupled to one or more components not shown.

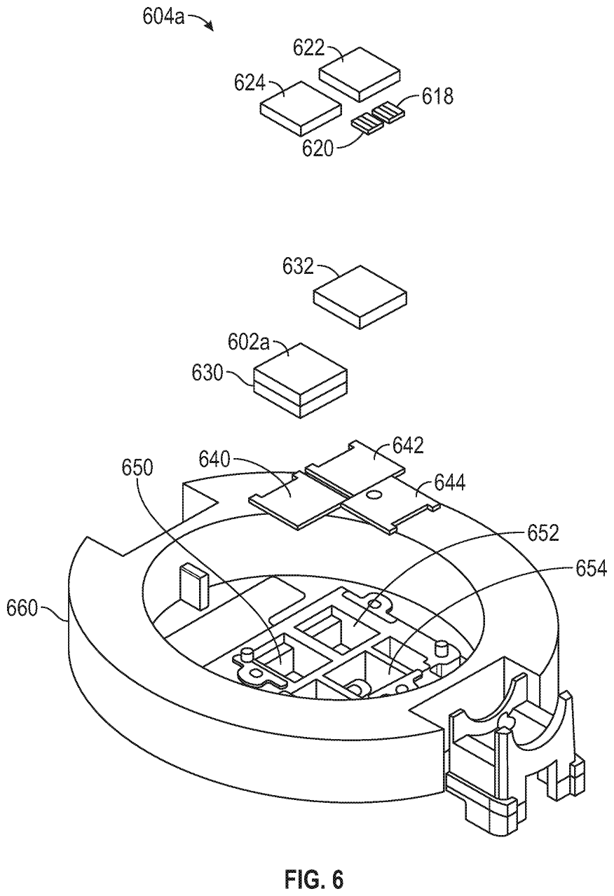

FIG. 6 is an exploded view of an inner housing 660 of the sensor head 510 illustrated in FIG. 5. FIG. 6 is an isometric view of the sensor head 604a with the upper housing and various electrical components removed to expose an inner housing 660. The inner housing 660 is contained within the housing. The inner housing 660 contains a sensor mount with a first detection well 652, a second detection well 650, and a light source well 654 formed therethrough. The first light detector 622 is mounted within the first detection well 652 and the second light detector 624 is mounted within the second detection well 650. The first and second light sources 618, 620 are mounted within the light source well 654. In an aspect, the first detection well 652, second detection well 650, and light source well 654 of the sensor mount are optically isolated from one another to ensure that light from the light sources 618, 620 does not reach the light detectors 622/624 without coupling through the skin of the patient. The separation between the two detection wells 652/650 ensures that the detected fluorescence signal from the exogenous fluorescent agent is distinguishable from the unfiltered excitation light, as described in detail above.

In one aspect, optically transparent windows 640, 642, and 644 are coupled within first detection aperture, second detection aperture, and light source aperture, respectively, to seal the apertures while also providing optically transparent conduits between the tissues and the interior of the sensor head 604a. In addition, diffusers 630, 632 are coupled over optically transparent windows 640, 642, and 644, respectively. The diffusers 630, 632 are provided to spatially homogenize light delivered to the tissues by light sources 618/620 and to spatially homogenize light detected by light detectors 622/624. In an aspect, the absorption filter 602a is coupled to the diffuser 630. In one aspect, an optically transparent adhesive is used to couple the absorption filter 602a to the diffuser 630. In aspects, one or more temperature sensors, e.g., thermistors, may be provided to measure temperature since temperature may affect the responsivity of a light detector such as a silicon photomultiplier. The measured temperature may be used to compensate for any effect on a light detector.

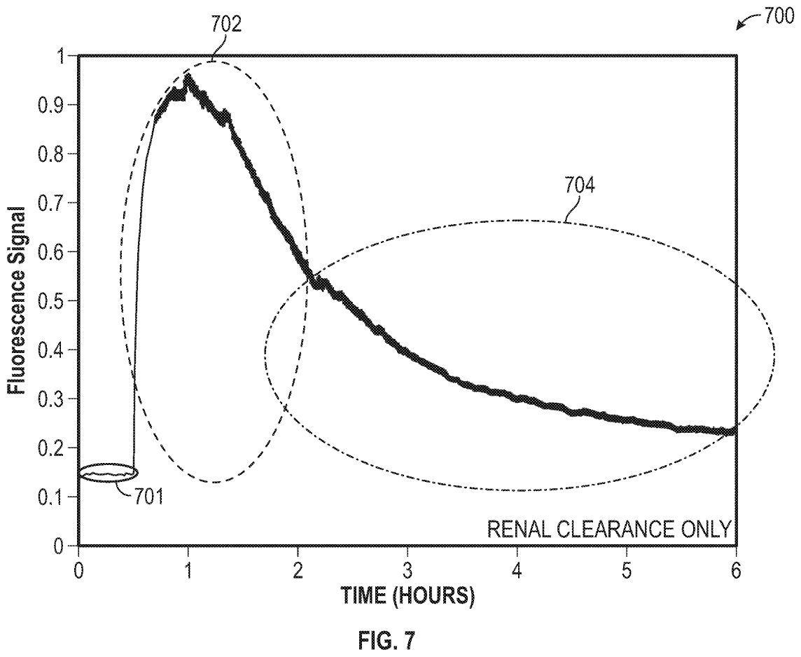

FIG. 7 illustrates a graph 700 of a measured intrinsic fluorescence (IF) signal profile during a diagnostic session, before and after administration of an exogenous fluorescent agent. After administration of the exogenous fluorescent agent, there are generally two phases where the body processes the exogenous fluorescent agent, a pre-renal decay dominated phase 702 and a renal decay dominated phase 704. The IF signal is also measured before administration of the exogenous fluorescent agent in order to establish a baseline period 701. The baseline period accounts for the autofluorescence from the bloodstream and tissue, and the baseline period is obtained to provide an accurate measurement of the IF signal without the exogenous fluorescent agent. For example, in FIG. 7, the baseline period is shown at the start of the fluorescence signal, at about 0.15. The baseline period ends when the exogenous fluorescent agent is administered to the patient. The actual waveform of the IF signal curve initially rises as the administered exogenous fluorescent agent establishes its distribution between the bloodstream and the tissue.

During the pre-renal decay dominated phase 702, the exogenous fluorescent agent continues to spread throughout the bloodstream and tissues increasing to a peak and then beginning to decay. During the pre-renal decay dominated phase 702, the rate of decay may not be adequately approximated as a single-time-constant exponential decay. At the renal decay dominated phase 704, the concentration of the agent in the blood and surrounding tissues stabilizes, such that the resulting IF signal decay curve reflects a predictable renal clearance rate by approximating a single-time-constant exponential decay. At a transition between the pre-renal decay dominated phase and the renal decay dominated phase, the rate of decay may briefly plateau or otherwise exhibit a change to the predictable single-time-constant exponential decay rate (e.g., fluctuation in the decay rate, deceleration, or other similar reactions).

To more accurately characterize and/or diagnose the patient's GFR, the renal decay dominated phase 704 is used since it can be characterized by the single-time constant exponential decay. To accomplish using as much of the renal decay dominated phase 704 in determining the patient's GFR, the (renal decay time constant (RDTC) may be calculated over a plurality of windows of time (e.g., 100 millisecond windows, 10 second interval windows, 1 minute interval windows, 5, 15, 30 or 45 minute intervals, or every hour, etc.), and determining based off a pre-defined threshold, when the RDTC is in the renal decay dominated phase 704. Once in the renal decay dominated phase 704, a plurality of RDTC measurements are taken across a plurality of windows of time, and may be averaged, compared, and reported until an End of Session is determined in accordance with methods described herein. The reported RDTC, is used to compute GFR based on an inverse relationship as detailed further below.

The IF signal may be characterized by the single-term exponential decay after reaching a renal decay dominated phase 704, according to equation 1: IF(t)=K₀×e^{-t/RDTC} (equation 1); wherein t is the time elapsed since the start of the renal decay dominated phase, defined at first as 0; K₀ is the value of the IF signal at that time; and RDTC is the single term-constant parameter representing the rate of the renal clearance of the exogenous fluorescent agent. The actual waveform of the curve initially rises as the newly injected agent establishes its distribution between the bloodstream and the tissue. This rise reflects the distribution of the exogenous fluorescent agent, during which the agent is absorbed into the bloodstream and begins to circulate systemically. Accordingly, the rate of change (slope) of the decay may be computed according to the following:

Slope of

$$IF ( t ) = \frac{d}{dt} IF ( t ) = \frac{- 1}{R DTC} \times {K}_{0} \times {e}^{- t / R D T C} = \frac{- 1}{R D T C} IF ( t ) \qquad ( equation 2 ) ,$$

where (d/dt) is the derivative with respect to time to provide the rate of change of decay at that moment t in time elapsed since the beginning of the renal decay dominated phase.

Then, once in the renal decay dominated phase, the RDTC may be calculated at any timepoint according to

$$RDTC = - \frac{IF ( t )}{( \frac{d}{dt} ) IF ( t )} , \qquad ( equation 3 )$$

since IF(t) is a known measurement and the slope is known per equation 2. To improve stability and reliability, an estimate of RDTC is determined over a defined time window t_{window} rather than at a single timepoint t. Accordingly, an estimate of RDTC may be determined at any timepoint according to

$${RDTC}_{estimated} = \frac{\vert average of IF \vert}{- 1 \times slope of IF} , \qquad ( equation 4 )$$

where the slope and average are calculated or computed over a time window t_{window}.

RDTC and nGFR are inversely related. The nGFR may thus be determined using the inverse relationship shown in equation 5:

$$nGFR ( ml / min ) = \frac{DFLT\_RABITO \_SLOPE \_GU \_MIN ( ml )}{60 min / hour} * \frac{1}{RDTC ( hours )} . \qquad ( equation 5 )$$

DFLT_RABITO_SLOPE_GU_MIN corresponds with an empirically derived constant. Additionally, an estimated nGFR may be similarly determined according to equation 6:

$$estimated nGFR = \frac{DFLT\_RABITO \_SLOPE \_GU \_MIN}{60} \times \frac{- 1 \times slope of IF}{\vert average of IF \vert} , \qquad ( equation 6 )$$

by inserting an estimation for the RDTC. Thus, as the exogenous fluorescent agent is processed by the body, a plurality of estimated nGFR may be provided over time and/or across windows of time. In aspects, the windows of time may not be fixed and can be adjusted based on whether a change in the RDTC is more or less than a predetermined threshold. In aspects, if the change in RDTC from one moment or window to the next is less than the predetermined threshold (e.g., a difference greater than 5%, 10%, 12%, 23%, 50%, or any suitable or desired difference), then the window may be widened until a more significant change in RDTC is determined. In aspects, this may be achieved by using the quality factors described herein. Accordingly, the above equation for estimated nGFR may be calculated using a range of windows (e.g. in 15-minute increments, starting at 1.5 hours and ending at 5 hours). In other words, calculations or computations may be performed from shortest to longest window, and a window cannot be used until there is sufficient data in the history to fill it. Accordingly, for each fitting window, a QF may be computed or calculated in association with the nGFR estimation as described in detail below in which the fitting window producing the best QF is selected using FIG. 8.

As the estimated nGFR is calculated for all of the fitting windows, either the fitting window that produces the best Quality Factor is chosen, or several fitting windows that have roughly the same Quality Factor are averaged as described with reference to FIG. 8.

In aspects, the fitting windows, may be elastic in size, rather than a fixed period. In aspects, a range of fitting windows may be used, including any range from 0 seconds to any desired increment of time, such that t is elastic in size and may vary. For example, in 15 minute increments, starting at 1.5 hours and ending at 5 hours. In another example, in 5 minute increments, starting at 1.5 hours and ending at 5 hours. In some examples, in 15 minute increments, starting at 1 hour and ending at 6 hours. The increments may be in 1, 2, 5, 10, and 20 minutes, but is not limited to fixed pattern increments. For example, the time increments may change from 10 minutes to 5 minutes, or every 4 minutes, or every 17 minutes, such that t is elastic in size.

The slope and average IF signal, and thus the RDTC, may be determined from the shortest to longest fitting window. In aspects, a fitting window may not be used or may be set aside until there is sufficient data in the fitting window to fill it, or only when a minimum quality factor validates the measurement data set spanning the fitting window. In aspects, a predetermined fitting window may have a window length or time period that is equal to or greater than a minimum fitting window length and/or equal to or smaller than a maximum fitting window length. In aspects a plurality of predetermined fitting windows are used to calculate a plurality of RDTCs, each RDTC of the plurality of RDTCs corresponding to one of the predetermined fitting windows, and each of the fitting windows also has a fitting window length that is equal to or greater than the minimum fitting window length and equal to or smaller than the maximum fitting window length.

FIG. 8 illustrates an example flow-chart of a multi-window GFR estimation algorithm for determining each predetermined window of a plurality of predetermined windows. As described herein, selecting the fitting window relies on calculating a QF for each predetermined window associated with an nGFR estimate. To ensure accurate measurement of the tGFR during the renal decay dominated phase, waiting until an ideal QF has been achieved in a fitting window may be required. However, if it is determined that this QF may not be attained during a session, a lower threshold allowing acceptable data may be used to begin reporting the tGFR.

Since QF correlates with tGFR accuracy, setting a QF threshold helps prevent reporting of inaccurate tGFRs. For a patient with constant tGFR, if QF is too low due to noisy data, reporting may be delayed until a window size is large enough, or the fluorescence decay is sufficient, to give an accurate tGFR. For a patient experiencing a change in tGFR, a shorter window size (which excludes the data prior to the change) may give a higher QF, allowing the tGFR change to be observed more rapidly than using a fixed, wide window. The tGFR may be calculated for the predetermined window, wherein the tGFR is calculated using data spanning the predetermined window. The calculated tGFR may then be reported.

In some aspects, two different quality factors may be computed. A first quality factor QF_{SNR} may be computed or estimated from a signal-to-noise ratio (SNR), which is based upon a Noise Model and Tolerance Model to convert the measured SNR, tGFR, and window size into QF_{SNR}. Thus, QF_{SNR} represents a quality estimated from the signal-to-noise ratio (SNR) that is measured as a coefficient of variation (CV), the standard deviation divided by the mean. The second quality factor, QFₗᵥₗ, may be computed or estimated from the signal level by comparing the current IF signal level to the IF signal at which the End of Session may be declared. Thus, QFₗᵥₗ represents a quality estimated from the raw signal level. QF_{SNR} and QFₗᵥₗ may be aggregated into a single QF value based on a lower value of the values of QF SNR and QFₗᵥₗ. In aspects, the values of QFₗᵥₗ, QF_{SNR}, and/or QF may be clipped to the range [0.0, 1.0] where 0.0 is the minimum acceptable value and 1.0 is the maximum acceptable value. QF_{SNR}, QFₗᵥₗ, and/or the aggregated QF may be constrained to a bounded numerical range, such as [0.0, 1.0], to prevent numerical instability or disproportionate weighting of extreme values. Other bounded ranges may be used depending on implementation. Constraining values within a defined range may improve robustness of fitting window selection and End of Session determination.

In some examples, the QF_{SNR} value may be calculated from a ratio of the measured window (Wₘₛᵣ) size to a “limiting” fitting window size (Wₗᵢₘ), where Wₘₛᵣ represents the window length used for generating the reported GFR value, and Wₗᵢₘ represents the limiting tolerance of the fitting window length. The limiting tolerance window length may be based upon a noise model for the combination of GFR and CV.

In some examples, a longer fitting window enables better averaging of noise and operation at a higher CV. Thus, the quality factor QF_{SNR} provides an assessment of how much longer a fitting window is being used than is necessary to account for noise to operate at a higher CV. Thus, the methods disclosed herein for fitting window selection using a QF advantageously reduces computational needs by looking for optimal fitting windows to calculate and report tGFR and when to end a session without doing so prematurely or excessively late. The QF_{SNR} may have two different threshold values. A first threshold value may be 1.0 indicating a desired level of the QF_{SNR}, and a second threshold value may be 0.5 indicating a good enough level of the QF_{SNR} at the beginning of the renal decay dominated phase to start predicting tGFR.

QF_{SNR} value may be computed using

$${QF}_{SNR} = \frac{{QF}_{t o l}}{1 - zs} \times ( \frac{{w}_{m s r}}{{w}_{lim}} - zs ) . \qquad ( equation 7 )$$

QFₜₒₗ is a predetermined lower tolerance limit, (e.g., QFₜₒₗ=RDTC_LOW_QUALITY_LIMIT). For example, the predetermined lower tolerance may be 0.5 on a scale of 0.0 to 1.0. In aspects, QFₜₒₗ may be any number from 0.0 to 1.0, such as 0.4, 0.35, 0.2, 0.17, or any desired lower limit if a different range for the quality scale is used (e.g., 1 to 10, or 1 to 100, etc., then a lower limit may be, e.g., 3, or 50, respectively). The value of QFₜₒₗ may correspond to determination of the RDTC at a tolerance bounding limit, or there below, at which an end of diagnostic session may be declared and determination of RDTC and tGFR is completed. In equation 7, ZS represents a ratio of Wₘₛᵣ/Wₗᵢₘ.

The QFₗᵥₗ value is calculated from the ratio of the current measured IF signal level (IFₘₛᵣ) to the “End-of-Session” signal level (IF_{EOS}) limit. IF_{EOS} is the IF signal level at which it is determined that the End-of-Session has been reached. The QFₗᵥₗ decreases as the IF signal level approaches the baseline level QFₗᵥₗ and thus measures how close the IF signal level is to some ‘limiting’ signal level (e.g., the IF_{EOS}). When the IF signal level limit is reached, the QFₗᵥₗ indicates the session should end so as not to go further below an IF signal level limit. This inhibits uncertainties in the baseline level from negatively impacting the accuracy of the measured tGFR.

Accordingly, one aspect of determining QFₗᵥₗ may be

$$Q {F}_{lvl} = 1 + ( \frac{1 - {QF}_{t o l}}{MG - 1} ) \times ( \frac{{IF}_{m s r}}{{IF}_{EOS}} - MG ) , \qquad ( equation 8 )$$

$$where MG = the ratio \frac{{IF}_{m s r}}{{IF}_{E O S}}$$

when a QF of 1 is reported.

Equations 7 and 8,

$${QF}_{SNR} = \frac{{QF}_{t o l}}{1 - z s} \times ( \frac{{w}_{m s r}}{{w}_{lim}} - zs ) and {QF}_{lvl = 1 +} \frac{1 - {QF}_{t o l}}{MG - 1} \times ( \frac{{IF}_{m s r}}{{IF}_{E O S}} - MG ) ,$$

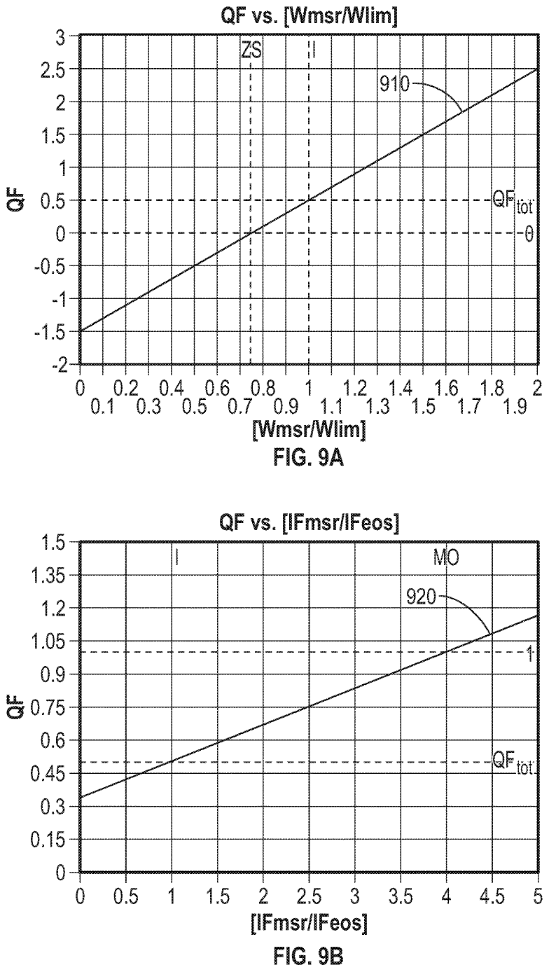

are linear for ratios ZS and MG, respectively, as shown in FIGS. 9A-B. In FIG. 9A, QF_{SNR} and Fitting Window ratio Wₘₛᵣ/Wₗᵢₘ are shown along Y-axis and X-axis, respectively, illustrating the linear progression 910 of QF_{SNR}, and in FIG. 9B, QFₗᵥₗ and IFₘₛᵣ/IF_{cos} are shown along Y-axis and X-axis, respectively, illustrating the linear progression 920 of QFₗᵥₗ. FIGS. 9A-9B are examples of the aforementioned linear relationships for a test tGFR diagnostic session.

With further reference to FIG. 8, a method 800 for determining tGFR fitting window selection using a QF includes the following steps. At block 802, the QF, including QF_{SNR} component and QFₗᵥₗ component is computed for a corresponding fitting window from one or more fitting windows or a plurality of predetermined fitting windows. At block 804, a first QF is calculated at each interval of measurement for which RDTC and tGFR is calculated using the measurement data set spanning the current fitting window. At block 806, the first calculated QF for a first predetermined fitting window is set as a base QF (bQF). The method then returns to block 802, and a next QF is determined for a next fitting window. Since the next QF is not the first QF, the method proceeds to block 808, where the next QF is compared to a predetermined value.

At block 808, the method includes determining if the next QF is greater than a first predetermined minimum value (PDV1). If the next QF is lower than the PDV1, then the method proceeds to block 810. Block 810 includes determining if the next QF is greater than the bQF. If at block 810 the QF is greater than the bQF, then the method proceeds back to block 806 and sets the next QF as the bQF, thus updating the bQF to the higher value. If at block 810 the QF is less than the bQF, then the method proceeds back to block 802, disregarding the next QF, and beginning the method over at block 802 to determine another next QF, and so forth.

If at block 808 the next QF is greater than the first predetermined minimum value, the method proceeds to block 812. The first predetermined minimum value may be the QFtol lower tolerance limit (e.g., 0.5) described above. Thus, at block 808, if the next QF is lower than the QFₜₒₗ, then the next QF is compared to the bQF at block 810, continuing as detailed above. When the next QF is more than the QFₜₒₗ or first predetermined minimum value, then the method proceeds to block 812.

At block 812, a difference between the next QF and the bQF is compared to a predetermined deviation value (PDV2) (e.g., 0.1). The value of PDV2 may be any acceptable predetermined maximum deviation value (e.g., 0.15, 0.05, 0.13, etc.). If, at block 812, the difference between the next QF and the bQF is greater than PDV2, then the method returns to block 806, and the next QF is set as the bQF, after which another QF is obtained for the next predetermined fitting window, repeating the method again from block 802. Thus, if the difference between the QF and the bQF is greater than a predetermined deviation, the bQF is updated with the value of the next QF.

At block 814, if the absolute difference between the next QF and the bQF is less than the predetermined deviation, then at block 816 the average of the QF and the bQF is taken.

At block 818, the method determines if additional fitting windows are needed based on the average of the QF and the base QF being less than a third predetermined value. At block 818, if the average of the QF and base QF is less than the third predetermined value, then blocks 802-816 are repeated until the average of the QF and the base QF are larger than a predetermined minimum value. If more predetermined fitting windows are needed based on the average of the QF and bQF being less than the third predetermined value, then the average of the QF and the bQF may be set as the bQF and the method repeats, until there are no more fitting windows, a limit of fitting windows is reached (e.g., a limit of 6 hours or 7 hours is reached), or the average of the QF and the bQF are greater than the predetermined minimum value.

If at block 818, the average of the QF and the bQF is greater than the third predetermined value, then no more fitting windows are needed, or a limit of fitting windows is reached, and the bQF is then reported at block 820.

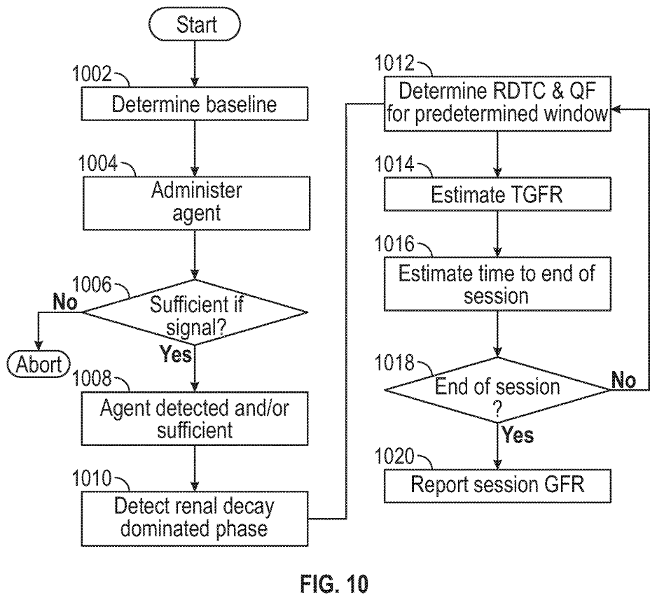

FIG. 10 illustrates a flow chart of a method for determining tGFR using fitting window selection in accordance with various aspects of this disclosure. As detailed above, a method for determining tGFR includes first determining a baseline of autofluorescence at block 1002. At block 1004, the exogenous fluorescent agent is administered. At block 1006, a determination is made whether there is a sufficient IF signal emitted by the exogenous fluorescent agent. If there isn't, then the session may be aborted if a predetermined period of time passes without a sufficient IF signal detected. In aspects, block 1006 may be performed at intervals until the predetermined period of time passes or the IF signal is sufficiently detected. Block 1008 includes, after determining if the exogenous fluorescent agent is detected and sufficient, providing the measurement data set collected to make the determination at block 1006 to detect renal decay dominated phase. At block 1010, renal decay dominated phase is determined according to the methods disclosed herein. When renal decay dominated phase is detected, a RDTC is calculated using the obtained measurement data set at block 1012. The RDTC corresponds to a predetermined window length that is equal to or greater than a minimum window length and/or equal to or smaller than a maximum window length. A QF corresponding to the predetermined window is computed at block 1012. The QF includes a QF_{SNR} component and a QFₗᵥₗ component. At block 1014, a tGFR is calculated for the predetermined window using the data spanning the predetermined window and may be reported. Blocks 1012 through 1016 continue for successive fitting windows using the calculated QF and methods disclosed herein for determining if additional fitting windows are needed based on the value of the QF, as described earlier with reference to FIG. 8.

Block 1016 estimates time to End of Session. If the End of Session is determined at block 1018, a session GFR is reported at block 1020. If not, then blocks 1012 through 1016 are repeated.

Block 1012 may estimate the RDTC in accordance with equations 1-3. The QFs may be determined in accordance with equations 6 and 7. In some aspects, in order to estimate the nGFR, a slope and average of IF signal may be used, as disclosed above, further using a 3-point linear regression estimate of the slope and average of the IF signal. In aspects, a fitting window may be divided into three sections, IF_{A}, IF_{B}, and IF_{C}. IF_{A} is an average over the first third of the fitting window, IF_{B} is an average over the second third of the fitting window, and IF_{C} is an average over the final third of the fitting window. A 3-point estimate of the slope of the IF signal in the fitting window may then be defined as (IF_{C}−IF_{A})/(⅔ of the fitting window). The average IF signal is (IF_{A}+IF_{B}+IF_{C})/3. In some aspects, the average of the slope may be IF_{B}. The average may then be used in equation 5 to determine the estimated nGFR. The slope determined using the 3-point method may be used as the slope in equation 4.

In various aspects, a correction factor may be used to minimize any errors introduced in estimating the nGFR. Thus, a corrected or actual nGFR may be determined by multiplying the estimated nGFR with the correction factor. The correction factor may be defined as G_{nGFR}(R). The correction factor depends on the ratio R=t_{window}/RDTC, where t_{window} is the fitting window calculated using the methods for determining a tGFR fitting window selection disclosed herein and the RDTC. In aspects, since the methods disclosed herein provide estimated nGFR and estimated RDTC, an iterative search may be calculated to determine a corrected nGFR. If given a fitting window that is too large, the ratio R may lead to an ambiguity in determining the corrected nGFR. Thus, a fitting window may be limited to a maximum ratio of the RDTC. Thus, a maximum fitting window may be used in any of the methods disclosed herein. In various aspects, a maximum fitting window length may be set to 1.5 times that of the last known good RDTC.

As a result of the RDTC-derived constraint on fitting window size, an anomaly in the reported GFR value may be generated during transitions from one nGFR calculation to another, when or if the GFR estimate is ambiguous. In those cases, long fitting windows may be used to report a ‘good quality’ GFR estimate by applying additional averaging across the IF signal in the fitting window. There may be more high signal/high SNR data from earlier in the session. Additionally, some short fitting windows may be used to report a ‘good quality’ by restricting their operation to a post-transition region of the decay curve (which would exclude the transitional data but would be expected to have a lower signal level and lower SNR). The methods for tGFR fitting window selection disclosed herein find the best or optimal fitting window. Moreover, because the GFR may oscillate in a transition region of the IF signal, averaging of the GFR over such a region mitigates such oscillations for a clearer determination of GFR.

In block 1014, the estimated tGFR may be reported. When the tGFR is estimated at short intervals, e.g., 10 seconds, each tGFR reported per interval may not be statistically different from prior or subsequent tGFR. To provide clinically relevant tGFR values, the tGFR may be reported at larger predetermined intervals, e.g., every 5 minutes, 15 minutes, 20 minutes, etc., as desired.

Blocks 1012 through 1018 may be repeated for a plurality of obtained measurement entries. Each measurement entry is obtained before or after administration of the exogenous fluorescent agent. A plurality of RDTCs is calculated. Each of the RDTCs corresponds to one of a plurality of predetermined windows, where each window has a window length greater than or equal to a minimum window length and equal to or smaller than a maximum window length. A plurality of QFs corresponding to each of the predetermined windows is computed. In aspects, the methods described herein may be used when computing the plurality of QFs.

When the plurality of QFs is more than the predetermined deviation from one another, one of the plurality of predetermined windows corresponding to a largest value of the plurality of QFs is selected. A tGFR is calculated from the one of the predetermined windows corresponding to the largest value of the plurality of QFs or calculated from the two or more predetermined windows corresponding to the two or more of the plurality of QFs. Again, the tGFR is calculated using data spanning the selected one of the predetermined windows or from the two or more predetermined windows corresponding to the largest value of the plurality of QFs. As disclosed above, a QF_{SNR} component is calculated from a ratio of a measured window size to a limiting window size, wherein the measured window size is a window length used to generate the present QF and the QFₗᵥₗ component is calculated from a ratio of an IF signal level to an End of a Session IF signal level. The limiting window size may be calculated from a noise model that determines a signal to noise ratio falls within a normal distribution.

In aspects, a short window may be used to capture a changing value of tGFR and a long window may be used to eliminate noise. The determination of the base QF provides for a measurement in which the noise is below a desired threshold.

In aspects, a renal decay dominated phase may be determined when one or more of the following occurs: QF_{SNR} is at or above high value; and QF_{SNR} is above the predetermined minimum value and is not increasing.

Block 1006 may include generating an intrinsic fluorescence (IF) signal representing a detected fluorescence intensity emitted by the exogenous fluorescent agent from within a diffuse reflecting medium. Next, at block 1008, it is determined if the IF signal includes a portion of premature fluorescence data. Upon determination of the portion of premature fluorescence data, a range of operable IF signals by filtering the IF signal associated with premature fluorescence data is identified. Block 1010 may include determining that a peak has been reached in the IF signal. Block 1010 may include determining if the QF_{SNR} has a value that is above a minimum reporting threshold. The minimum reporting threshold provides sufficient data quality to predict the tGFR. In aspects, block 1010 may include determining that the IF signal is in a renal decay dominated phase based upon passing of the peak and the QF_{SNR} having a value above the minimum reporting threshold.

In some examples, for determining when an initial renal decay dominated phase is reached, measured optical signals may be used by an algorithm, The algorithm may perform the following steps.

In a first step, a peak is detected in the IF curve by waiting until the peak occurs. The peak may be detected by comparing the plurality of RDTC values from at least two segments until all the plurality of RDTC values correspond to positive values.

In the next step, upon detecting the peak, the GFR is calculated or computed using the multi-window approach described herein. As part of the computation, a quality factor (e.g., QF_{SNR}) is derived from the SNR, and the value of which is tracked and used to decide if renal decay dominated phase has been reached. In particular, if QF_{SNR} rises above 1.0, it may be declared that initial renal decay dominated phase is reached, which represents a situation where the quality of the measured GFR approximate the actual GFR. Further, if QF_{SNR} has not reached 1.0 but has reached 0.5 and has stopped increasing, then initial renal decay dominated phase may be declared where the quality is as good as it is going to get. However, if QF_{SNR} never reaches 0.5, then the initial renal decay dominated phase may not be declared. Renal decay dominated phase may be declared after a calculated delay after initial renal decay dominated phase, as described herein. If a minimum acceptable QF threshold is not achieved during a session, entry into the renal decay dominated phase may not be declared. Reporting of tGFR values may be suppressed when signal-to-noise ratio remains insufficient.

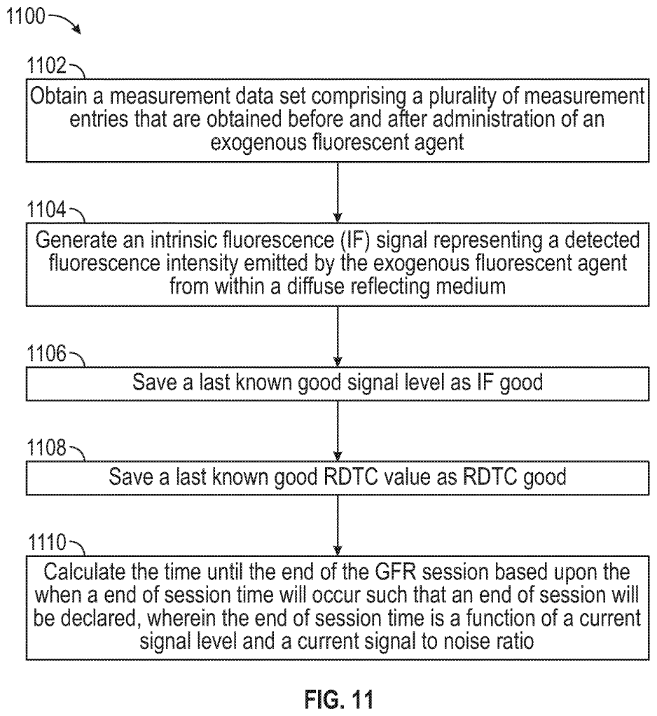

With reference to FIG. 11, this disclosure further provides a method 1100 for predicting a time until an end of a GFR session using an exogenous fluorescent agent. The method may be implemented, for example, at block 1018. A time until end of a GFR session may be predicted based upon a current signal level and a current SNR. The current signal level may be determined by using the most recent valid IF level. The IF level may be estimated by the RDTC by extrapolation to the IF level at which the End of Session may be declared. In aspects, a delay may be added to the End of Session time estimates to account for the delay built into an End of Session determination method, and the shorter of the two-time estimates may be reported as the End of Session prediction. A time until End of Session prediction provides the clinician with an estimate of how much longer tGFR measurements will be reported during the session.

The method 1100 for predicting a time until an end of a GFR session includes, at block 1102, obtaining a measurement data set that includes a plurality of measurement entries recorded before and after the administration of the exogenous fluorescent agent. The method may use the measurement data set from earlier blocks in FIG. 10. An intrinsic fluorescence (IF) signal is generated, at block 1104, to represent the detected fluorescence intensity emitted by the exogenous fluorescent agent within a diffuse reflecting medium. The method further includes saving, at block 1106, a last known good signal level as IF_{good} and saving, at block 1108, a last known good RDTC value as RDTC_{good}. The time until the end of the GFR session is then calculated, at block 1110, based on when an End of Session time will occur, wherein the End of Session is declared as a function of the current signal level and the current signal-to-noise ratio.

The time until an end of GFR session method may be used to predict how long until the IF signal is so low that an accurate nGFR estimate cannot be made. That is, it is used to predict at what point in the future it is expected that the “End of Session” will be declared. Much like the Quality Factor described above, this prediction is based on two factors: (1) the current signal level, and (2) the current SNR. For the signal level, the algorithm makes use of the End of Session fluorescence signal level IF_{EOS} described above.

Using IF_{good}, and RDTC_{good}, the time until the End of Session based on the signal level, t_{EOS, lvl}, is calculated. This calculation is performed using the following equation:

$${t}_{EOS , l v l} = - 1 \times {RDTC}_{g o o d} \times ln \frac{{IF}_{E O S}}{{IF}_{g o o d}} + \frac{1}{2} {W}_{min} .$$

In this equation, Wmin represents the minimum fitting window length. An adjustment is made by multiplying Wmin by (½) and is included to account for a delay between detecting the “low signal” condition and actually declaring the End of Session. This adjustment ensures that the method compensates for the fraction of the minimum window length that elapses during this waiting period.

For the signal-to-noise ratio (SNR), an empirical model is employed to estimate how the SNR evolves over time. This model is based IF_{good}, RDTC_{good}, CV_{good}, W_{good}, and GFR_{good}. A noise model may be used to estimate a limiting coefficient of variation CVₗᵢₘ for the maximum available fitting window at GFR_{good}. This determines the largest coefficient of variation that could be handled if the fitting window were expanded to its maximum size.

The estimated time until the End of Session based on SNR, t_{EOS, lvl}, is calculated

$${t}_{EOS , SNR} = - 1 \times {RDTC}_{g o o d} \times ln ( \frac{{k}_{c x}}{{IF}_{g o o d}} ) + {W}_{a d j} .$$

First, the noise is calculated as σ=CV_{good}×IF_{good}; Next, the parameters

$${k}_{c x} = {( \frac{{CV}_{lim}}{σ} {k}_{c p} )}^{\frac{1}{fPOWER - 1}}$$

and k_{cp}=IF_{good}^{fPOWER} are derived using empirically defined relationships. fPOWER is also an empirically-derived constant. This calculation provides an ad hoc, empirical estimate of how the SNR is expected to evolve over time.

To further refine the estimation, an adjustment term, W_{adj}, is included to account for the delay between detecting the low SNR condition and declaring the End of Session. The adjustment term is calculated as follows: W_{adj}=min (fMARGIN_FRAC×(W_{good}+fMARGIN_MIN), fMARGIN_FRAC×Wₘₐₓ). Here, Wₘₐₓ represents the maximum fitting window size, and the adjustment is scaled by the constants fMARGIN_FRAC and fMARGIN_MIN, which are empirically derived parameters. This adjustment ensures that the delay in declaring the End of Session is appropriately accounted for, even under varying signal and noise conditions.

Once both the SNR and signal level estimates are determined, the estimated time until the End of Session is defined as: t_{EOS}=min(t_{EOS,lvl}, t_{EOS, SNR}), or the minimum (lower value) of t_{EOS, lvl} or t_{EOS, SNR}. In aspects, a temporal smoothing may be applied to an estimated so that the time under the end of a GFR session is calculated as reported t_{EOS}[n]=(1−0.03)×t_{EOS}[n−1]+0.03×t_{EOS}[n].

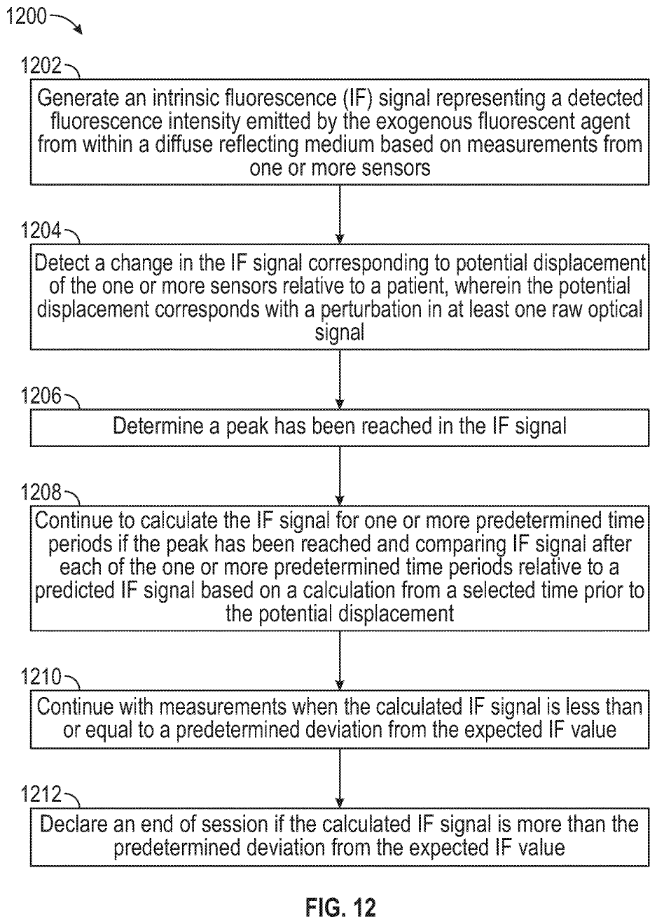

With reference to FIG. 12, in accordance with further aspects of this disclosure, a displacement detection method is provided to identify significant changes in tissue properties associated with a shift in sensor placement. Since the fluorescence signal is a response to the stimulus, such displacements have little effect if the tissue changes affect two or more diffuse reflectance signals (e.g., a blue DR signal and green DR signal) to the same extent; but they have a large effect if the changes affect the two or more DR signals (e.g., blue and green DR signals) differently. Hence, the displacement detection algorithm is designed to detect differential changes in the DR signals, (e.g., blue and green signals). Differential changes may indicate a displacement. A predetermined period of time may be allowed for signal recovery, and may depend on whether the displacement was flagged before or after the renal decay dominated phase is detected.

Further, an adhesive used to hold the sensor in place is adequate to inhibit slow drifts in position over time. However, sensor “displacements” tend, therefore, to occur as sudden shifts associated with high instantaneous strains on the sensor. Hence, the displacement detection algorithm specifically looks for such differential changes over a fairly short fitting window.

Additionally, a sensor that is displaced due to a momentary strain may be only elastically displaced (e.g. by a momentary stretching/twisting of the skin, rather than by a release in the adhesive) and may, therefore, recover its original position once the strain is removed. Hence, the displacement detection algorithm also allows for the possibility that the signals will recover from such a differential change.

If displacement is detected, in accordance with methods described herein, and the IF signal does not return to a sufficient IF signal level, the session may terminate after a predetermined period of time where the IF signal level is insufficient. The predetermined period of time may be shorter before renal decay dominated phase is detected, but longer after.

In block 1202, method 1200 generates an intrinsic fluorescence (IF) signal representing a detected fluorescence intensity emitted by the exogenous fluorescent agent from within a diffuse reflecting medium based on measurements from one or more sensors. In block 1204, method 1200 detects a change in the IF signal corresponding to potential displacement of the one or more sensors relative to a patient, wherein the potential displacement corresponds with a perturbation in at least one raw optical signal. In block 1206, method 1200 determines a peak has been reached in the IF signal. In block 1208, method 1200 continues to calculate the IF signal for one or more predetermined time periods if the peak has been reached and comparing IF signal after each of the one or more predetermined time periods relative to a predicted IF signal based on a calculation from a selected time prior to the potential displacement. In block 1210, method 1200 continues with measurements when the calculated IF signal is less than or equal to a predetermined deviation from the expected IF value. In block 1212, method 1200 declares an End of Session if the calculated IF signal is more than the predetermined deviation from the expected IF value.

Sensor displacement events may be categorized as elastic (temporary mechanical strain or skin deformation) or permanent (e.g., sustained sensor shift or adhesive failure). Elastic displacement events may result in transient perturbations of IF signal or diffuse reflectance signals that recover within a predetermined observation window. Permanent displacement events may produce sustained deviation beyond a predetermined threshold and may result in termination of the diagnostic session. Differentiation between temporary and sustained displacement reduces unnecessary session termination while preventing reporting of corrupted data.

In aspects, method 1200 may continue to calculate the IF signal for a shortened predetermined period of time if the peak has not yet been reached and comparing IF signal after the shortened predetermined period of time relative to a predicted IF signal based on a calculation from the selected time prior to the potential displacement. The method 1200 may include declaring an End of Session if the calculated IF signal is more than the predetermined deviation from the expected IF value.

The method 1200 includes providing a measurement data set comprising a plurality of measurement entries comprising at least one diffuse reflectance signal at an excitation wavelength of the exogenous fluorescent agent (DRex), a diffuse reflectance signal at an emission wavelength of the exogenous fluorescent agent (DRem), and a fluorescence emission (Flr) signal. The method 1200 includes estimating a first signal median within a first window and estimating a second signal median within a second window. The method 1200 further includes calculating a difference between the first signal median and the second signal median for each of DRex, DRem, and Flr, and normalizing the difference to a corresponding signal level. The method 1200 includes calculating a first absolute difference by taking an absolute value of a difference in the DRex minus a difference in DRem, and calculating a second absolute difference by taking an absolute value of a difference in the Flr minus a difference in DRex. The method 1200 includes declaring the potential displacement if both the first absolute difference and the second absolute difference are greater than a nominal threshold.

In further aspects, the method 1200 includes creating a reference IF value from IF data at a predetermined time prior to the potential sensor displacement. The method 1200 further includes projecting the IF signal over a predetermined time interval using a last known RDTC value and determining a difference between the projected IF signal and an actual IF measurement at the potential sensor displacement. The method 1200 includes declaring the potential sensor displacement to be insignificant or temporary if the actual IF measurements are within a predetermined percentage of the projected IF measurements, and declaring the potential sensor displacement to be significant or permanent if the actual IF measurements are outside a predetermined percentage of the projected IF measurements.

The projecting of the IF signal may use the following formula: IFₚᵣⱼ(t)=IF_{ref}e^{-t/RDTC}.

A fitting window of between ten and thirty minutes is used to determine whether the potential sensor displacement is temporary or permanent. For each window of ten minutes spanning a maximum time of thirty minutes, the following may be calculated: ΔIF=(IF−IFref)/IFref; and ΔIFnom=(IFprj−IFref)/IFref. If |ΔIF−ΔIFnom|>(4.5×Last CV), the method includes declaring that the sensor is currently displaced. If the IF signal does not recover, and the displacement lasts longer than a predetermined maximum waiting period, such as 30 minutes, the session may be ended. In aspects, if the displacement is temporary, the tGFR determinations or IF signal data from the displacement period may be discarded.

Additional technical aspects of a method for detecting sensor displacement is disclosed as follows. In some examples, the displacement detection algorithm operates on DRem2 (SiPM2 Green), Flr2 (SiPM2/Blue) and DRex2 (Heterogeneity-corrected SiPM1 Blue) channels, with DRex compared to DRem2 (e.g., a verification that the Heterogeneity-Corrected Blue signal tracks the green signal) and with Flr2 compared with DRex2 (e.g., a verification that the blue signals on both detectors track each other). For each of those signal pairs, the method may be as follows:

In the first step, the signal median within QF_{SNR} 2-second windows with a 2-second gap between them may be estimated. Next, the difference between those two median values may be computed. The two median values are thus used to estimate the amount of change in the signal (ΔDRem2, ΔDRex2, ΔFlr2) over a short interval, for example, about a 4 to 6 second interval. Other intervals may be used, such as by way of examples, 3 to 5 seconds or 4 to 7 seconds, or larger intervals. The delta values may be normalized to the signal level so that changes can be assessed on a relative scale (e.g., as a percentage change).

Further, an absolute difference in the normalized deltas between the two channels may be computed as Dif1=|ΔDRex2−ΔDRem2| and Dif2==|ΔFlr2−ΔDRex2|. IF both differences Dif1 and Dif2 exceed a nominal threshold value, for example, 20%, a potential displacement may be declared.

As described herein, a process for detecting a potential displacement of the sensor position on the skin that is sufficiently disruptive to invalidate the baseline calculated as described herein may be deferred until a cycle of a method illustrated by FIG. 10 is completed and/or validated. Confirmation (or rejection) of a sensor displacement may be done using the IF signal coming from the Outlier Rejection filter that applies a median filter to the decimated IF signal which efficiently removes gaussian white noise and short-term signal excursions without distorting the IF signal. The process begins when the underlying signal processing algorithm reports a potential displacement; it ends when the displacement is determined as ‘significant’ or ‘permanent’ (at which point the session is ended) or ‘insignificant’ or temporary (at which point the session is continued).

An example signal processing algorithm may use data from the sample history to record a reference IF value just before the potential displacement was detected and use the last known-good RDTC value to project how much the IF curve ought to have decayed during the time interval on the validation. If the actual IF measurements are close enough to the projected IF decay that there does not appear to be any observable perturbation, the potential displacement may be declared as insignificant or temporary.

Accordingly, whether a significant or temporary perturbation in the raw optical signals has had an equivalently significant or permanent effect on the IF signal calculated (in a roundabout way) from those raw signals may be evaluated or checked. Further, the ‘potential displacement’ indicator in the underlying signal processing module may be cleared. Accordingly, any new report of a potential displacement may result in an appropriate extension of the overall observation window.

As described herein, the reference IF value (IF_{ref}) may be the IF value from a sample history taken at a predetermined period for displacement comparison, for example, 5 minutes, before the original declaration of a potential displacement. The IF decay may be projected forward from that value/time (to IFₚᵣⱼ) using a standard single-exponential decay curve model using IFₚᵣⱼ(t)=IF_{ref}e^{-t/RDTC}.

In some examples, a test for a significant deviation may be performed on the current samples within a fitting window that spans a displacement testing period. The displacement testing period may be of any range, for example, from 0 minutes to 30 minutes after detection of the potential displacement. The 0-minute delay allows time for any perturbations to work their way through the outlier rejection filter, while a 30-minute maximum time of the range provides a reasonable amount of time for the signal to recover before a permanent disruption may be declared.

In some examples, for each new IF sample within the observation window, displacement may be computed using the following equations.

$$Δ IF = ( IF - {IF}_{r e f} ) \div {IF}_{r e f}$$

$$Δ {IF}_{nom} = ( {IF}_{p r j} - {IF}_{r e f} ) \div {IF}_{r e f}$$

$$Currently Displaced = \vert Δ IF - Δ {IF}_{nom} \vert > ( 4.5 \times Last CV )$$

Accordingly, if the current sample represents a displacement if it is more than 4.5 standard deviations from where it is expected to be based upon the last known-good CV value representing the signal standard deviation. In the case when the signal consistently ‘not displaced’ for a full minute (for example, 6 samples at the 0 second decimated sample interval), then the displacement may be declared as ‘insignificant’ or temporary, and the validation process may be ended. If, at the end of the 30-minute validation period, if the displacement is not declared as ‘insignificant’ or temporary, then a sensor displacement may be declared, and the session may be ended. During the observation process, samples may be cleared by invalidating them from the sample history during the intervals where the samples that are currently displaced’ are observed. Accordingly, any potential corruption of the samples may be prevented from contributing noise to the GFR estimate.

In some examples, an End of Session algorithm can be interpreted as the inverse of the agent sufficient algorithm. Purpose of the End of Session algorithm is to analyze the measured fluorescence signal and determine if the signal-to-noise is so low that an accurate nGFR estimation cannot be made. In particular, the End of Session may be based on the time once the QF falls below a limiting tolerance level of RDTC_LOW_QUALITY_LIMIT, which may be, e.g., 0.5. As described herein, the QF is lower of the two values-QF_{SNR} and QFₗᵥₗ. The QF_{SNR} is calculated based on the SNR and the QFₗᵥₗ is computed based on the signal level.

In some examples, for QF_{SNR}, based on the noise model, the limiting tolerance of RDTC_LOW_QUALITY_LIMIT is reached when the window size cannot be increased enough to compensate for the apparent noise. For QFₗᵥₗ, reaching the limiting tolerance of RDTC_LOW_QUALITY_LIMIT depends on the IF signal at the End of the Session. IF_{EOS} is computed according to IF_{EOS}=TRACER_INVALID_DECAY_RATIO×IF_{ref}.

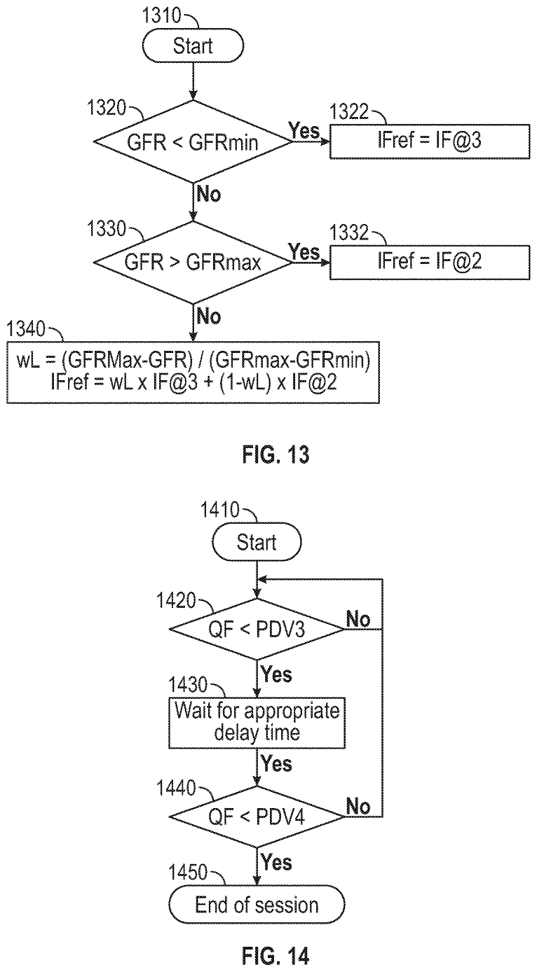

With reference to FIG. 13 and FIG. 14, this disclosure further provides a method for detecting an end of Glomerular Filtration Rate (GFR) using an exogenous fluorescent agent. In some examples, an End of Session detection method can be interpreted as the inverse of the agent sufficient algorithm. Purpose of a method for detecting an end of GFR diagnostic session is to analyze the measured fluorescence signal and determine if the signal-to-noise is so low that an accurate nGFR estimation cannot be made.

A quality factor (QF) is computed for a predetermined window of a measurement data set, which comprises a plurality of measurement entries obtained before or after the administration of an exogenous fluorescent agent. The QF includes both a QFsnr component and a QFlvl component. Using data spanning the predetermined window, a transdermal Glomerular Filtration Rate (tGFR) is calculated. The lower of QFsnr or QFlvl is assigned as the end determining QF, and an End of Session is declared once this value falls below a limiting tolerance level—a threshold below which noise is no longer considered insignificant.

A measurement data set may be obtained, comprising a plurality of measurement entries recorded before and/or after the administration of an exogenous fluorescent agent. From this data, an intrinsic fluorescence (IF) signal is generated, representing the detected fluorescence intensity emitted by the exogenous fluorescent agent within a diffuse reflecting medium. At a first predetermined time after the agent is detected, a first recorded IF reference signal is set, followed by a second recorded IF reference signal at a later predetermined time. Using these reference signals, an invalid decay ratio is calculated based on the smaller ratio of either the current IF signal to the first recorded reference signal or the current IF signal to the second recorded reference signal. If the invalid decay ratio falls below a predetermined End of Session value, a possible End of Session is declared.

It is determined whether the IF signal includes a portion of premature fluorescence data. Upon determination of such a portion, a range of operable IF signals is identified by filtering out the portion of the IF signal associated with the premature fluorescence data. The first recorded IF reference signal and the second recorded IF reference signal are both set to occur after a peak in the IF signal. If the End of Session is determined based on the QFₗᵥₗ component of the QF, the End of Session is reset if the QF rises above the limiting tolerance level during a time delay. The time delay can be a predetermined time delay or a calculated time delay.

If the End of Session is determined based on the QF_{SNR} component of the QF, a time delay is calculated based on one of the following: if the QF_{SNR} is below a predetermined limiting tolerance level, a first time delay is set; if the QF_{SNR} is above the predetermined limiting tolerance level, a second time delay is set. The End of Session is reset if the QF rises above the limiting tolerance level during either the first time delay or the second time delay.

In further technical detail, the End of Session may be based on the time once the QF falls below a limiting tolerance level, or RDTC_LOW_QUALITY_LIMIT, which may be, e.g., 0.5. As described earlier, the QF may be defined as the lower of the two QF component values−QF_{SNR} and QFₗᵥₗ. The QF_{SNR} is calculated based on the SNR and the QFₗᵥₗ is computed based on the signal level.

FIGS. 13 and 14 illustrate an example flow-chart of computing or calculating IF signals, where a portion of the method starts at block 1310. At block 1320, the method determines if the GFR is less than a GFRmin. If it is not, the method can proceed to block 1330 to determine if GFR is greater than GFRmax. As shown in the flow-chart, the End of Session is reached when the IF signal falls below a percent of a reference IF signal, IF_{ref}, termed a TRACER_INVALID_DECAY_RATIO. The value of TRACER_INVALID_DECAY_RATIO may be, for example, 12% of IF_{ref}. The reference IF signal may be calculated from the IF signal levels recorded at period of time, such as, 2 hours, after the agent has been detected (IF@2) at block 1322 and/or at 3 hours after the agent has been detected (IF@3) at block 1332, or other chosen intervals. As shown block 1322 corresponds to the IFref when GFR is less than GFRmin at block 1320. As shown block 1332 corresponds to the IFref when GFR is greater than GFRmax at block 1330. The chosen level may depend on the most recent estimated GFR values, as shown in FIG. 13.

As shown in block 1340, during the baseline period, the IFref is set to be fSIG_QUAL_MARGIN multiplied by the noise level in the IF signal. For these calculations, the raw IF signal is not used, but rather it is first passed through a temporal filter to reduce noise according to IF[n]=(1−TRACER_INSUFFICIENT_COEF_NEW)×IF[n−1]+TRACER_INSUFFICIENT_COEF_NEW×IF[n]. TRACER_INSUFFICIENT_COEF_NEW is an empirically obtained value.

Further the method may have an additional start at block 1410, where a delay time, block 1430, may be added to time when, at block 1420, the QF goes below PDV3 or RDTC_LOW_QUALITY_LIMIT, which establishes the End of Session time. The purpose of this delay, block 1430, is to compensate for situations like when a mere noise spike is causing the QF to drop below RDTC_LOW_QUALITY_LIMIT or PDV4 momentarily at block 1440, and when for the QF_{SNR} calculation, where it is temporarily below the SNR threshold because there is an actual change in GFR happening that should be caught up to. The amount of delay time that is added may depend on the reason for which the End of Session was declared. Baseline drift of the IF signal may increase over longer diagnostic sessions. Because lower GFR values may be associated with longer session durations due to slower renal clearance, baseline drift effects may be more pronounced at lower GFR values. QFₗᵥₗ threshold behavior and/or End of Session criteria may therefore be adapted as a function of estimated GFR to account for session-length-dependent baseline variation.

As described herein, if the End of Session (EOS) was reached because QFₗᵥₗ<RDTC_LOW_QUALITY_LIMIT, then the delay time may be 11% of the minimum fitting window. IF EOS was reached before QF_{SNR}<RDTC_LOW_QUALITY_LIMIT, then if the signal is below 125% of the “low signal” threshold (close to low signal), then the delay time is 11% of the minimum fitting window, and if the signal is above 125% of the low signal, then the delay time may larger of the following two values, 60% of the most recent fitting window time or ⅙ of the most recent RDTC, capped, for example, at 3.5 hours.

Regardless of the delay time, in some examples, the QF<RDTC_LOW_QUALITY_LIMIT condition may be checked continuously during the delay period and, if it is found that QF>RDTC_LOW_QUALITY_LIMIT, then EOS is not declared, at block 1450 as illustrated in FIG. 14.

In aspects, this disclosure also provides a method of determining a waiting period length before an exogenous fluorescence agent can be re-injected into a patient for GFR monitoring. In aspects, consecutive GFR measurements may be taken. In order to ensure that injection of additional exogenous fluorescent agent does not interfere with a current GFR session, the following method may be used to determine an appropriate period for reinjecting the agent. The method optimizes the re-injection of the agent by ensuring the agent is not injected too early to cause interference but not too late to minimize a blind spot when a new tGFR reading is available.

The method includes determining a baseline level of auto-fluorescence prior to injection of the exogenous fluorescent agent. Another step of the method includes generating an intrinsic fluorescent (IF) signal. The method includes calculating a transdermal Glomerular Filtration Rate (tGFR). Based upon the tGFR, the method includes estimating a residual tracer or agent concentration. The method includes determining a calculated time, after which the residual agent concentration does not cause significant bias on the tGFR, wherein the calculated time is used as when a second injection of the exogenous fluorescent agent can be made.

The method includes determining a wait time from the tGFR and/or from an estimated GFR (eGFR). The calculated time is determined by minimizing the period of time from a last tGFR from a first injection of the exogenous fluorescent agent to a second injection of the exogenous fluorescent agent.

In aspects, when an IF_{EOS} is determined, an agent cleared IF level may be determined according to IF_{clr}=CLRMARGIN×IFEOS, where CLRMARGIN is an empirically derived constant. IF_{clr} represents the IF signal below which the agent is expected to be sufficiently cleared such that an additional injection of the agent will not affect the GFR measurements of the first GFR session. Before the agent is injected, IF_{clr} may be a baseline value of the IF signal. Once the End of Session has been declared, then IF_{clr} is updated according to: IF_{clr}=F/RS×√(K×estimated RDTC)×IF_{ref} wherein, F, RS and K are constants. These constants may be empirically derived from population data or calibration datasets and may be pre-stored within the device firmware. Additionally, in at least one instance CLRMARGIN can be a scaling factor that defines the “agent cleared” IF level relative to the End of Session IF level. Before agent injection, IF_{clr} is set equal to the baseline IF value. After End of Session is declared, the clearance threshold may be updated using the model described herein in regards to F, RS, and K. The constants F, RS, and K may are derived from a tolerance model that accounts for the relationship between GFR measurement tolerance and residual agent concentration. The formula IF_{clr}=F/RS×√(K×estimated RDTC)×IF_{ref} allows the tolerance for residual agent to increase as RDTC increases (for example, as GFR decreases). Without this relationship, the time to agent clearance would grow exponentially at very low GFR values. IF_{ref} is determined as shown in FIG. 13 and described herein. A time until agent clearance may be estimated according to t_{clearance}=−1×estimated RDTC×ln(IF_{clr}/IF_{good})−(t_{now}−t_{good}) wherein t_{now} is the current time, and t_{good} is the time at which the last good IF and RDTC values were recorded (e.g., when the last time QF>RDTC_LOW_QUALITY_LIMIT). Determination of the time until agent clearance may be performed when an End of Session is declared.

As shown in FIG. 14, when the End of Session is identified at block 1450, the session GFR gets reported. The session GFR is the weighted sum of all the GFR values that were previously reported, with the weighting equal to the reported QF. For a given GFR, if the QF>RDTC_LOW_QUALITY_LIMIT, the aggregate value may be updated based upon LastGFRTime-Current time; CumulativeGFR=CumulativeGFR+nGFRxQF; CumulativeQF=CumulativeQF+QFxQF; CumulativeWeight=CumulativeWeight+QF.

At the End of Session, the final value may be computed as SessionGFR=CumulativeGFR/CumulativeWeight, and Session QF=CumulativeQF/CumulativeWeight. The fitting window time reported for the session GFR is the span from the earliest data recorded into the history (during renal decay dominated phase) and the LastGFRTime, as calculated above, which is the last time at which a valid GFR report was generated. Additional cumulative parameters may be maintained to generate a corresponding SessionQF. Cumulative weighted aggregation reduces oscillatory behavior and provides a clinically stable reported session value.

In some examples, an algorithm used to predict how long until the IF signal is so low that an accurate nGFR estimate cannot be made is used to predict at what point in the future is it expected that the End of Session will be declared. As shown herein, similar to the Quality Factor, this prediction may also be based on two factors: the current signal level, and current SNR. For the signal level, the End of Session fluorescence signal level IF_{EOS} described herein may be used. Then, based on the last known good signal level (IF_{good}) and the last-known good RDTC (RDTC_{good}), t_{EOS, lvl} may be calculated as

$${t}_{EOS , lvl} = - 1 \times {RDTC}_{g o o d} \times ln \frac{{IF}_{E O S}}{{IF}_{g o o d}} + \frac{1}{2} {W}_{min} ,$$

where Wₘᵢₙ is the minimum fitting window length. The adjustment by Wₘᵢₙ/2 is intended to compensate for the fact that a waiting period for some fraction of a minimum window length after detecting the low signal condition before declaring End of Session is required.

For the SNR, an example empirical mode is used in which for a given last-known good signal level (IF_{good}), CV (CV_{good}), RDTC (RDTC_{good}), GFR (GFR_{good}), and fitting window size (W_{good}), the noise model may be used to estimate the limiting CV (CVmin) for the maximum available fitting window at GFR_{good} so that a bigger CV could be handled when the fitting window is expanded to the maximum size.

The estimated time until the End of Session may be computed as follows: determining:

$${t}_{EOS , l v l} = - 1 \times {RDTC}_{g o o d} \times ln \frac{{IF}_{E O S}}{{IF}_{g o o d}} + \frac{1}{2} {W}_{min} and$$

$${t}_{EOS , SNR} = - 1 \times RDT {C}_{g o o d} \times ln ( \frac{{k}_{c x}}{{IF}_{g o o d}} ) + {W}_{a d j} .$$

The term k_{cx} is determined according to

$${k}_{c x} = {( \frac{{CV}_{lim}}{σ} {k}_{c p} )}^{\frac{1}{fPOWER - 1}} ,$$

wherein k_{cp}=IF_{good}^{fPOWER} and fPOWER is an empirically-derived constant. σ is defined according to: σ=CV_{good}×IF_{good}. Additionally, W_{adj}=min(fMARGIN_FRAC×(W_{good}+fMARGIN_MIN), fMARGIN_FRAC×W_{MAX}. The End of Session is a minimum of t_{EOS, lvl} or t_{EOS, SNR}. The value W_{good} is the last known good fitting window size. The value Wₘᵢₙ is the minimum fitting window size. CVₗᵢₘ is a coefficient of variation at a limiting window size, defined as a function of RDTC, tGFR and/or a fitting window size. CVₗᵢₘ is not a fixed value and may fluctuate. CV_{good} is a coefficient of variation at the last known good fitting window. fMARGIN_FRAC and fMARGIN_MIN are empirically derived parameters, defined as approximately 0.6 and 30.0, respectively. In the above equations, fPOWER is an empirically derived constant referenced above, which corresponds to an ad hoc, empirical estimate of how the SNR is expected to evolve over time.

The final adjustment term (W_{adj}) is intended to compensate for the delay between detection of the low SNR condition and when the EOS is actually declared. W_{adj} may be calculated as follows: W_{adj}=min (fMARGIN_FRAC×(W_{good}+fMARGIN_MIN), fMARGIN_FRAC×Wₘₐₓ) where Wₘₐₓ is the maximum window size.

Once both the SNR and signal level estimates have been made, the estimated time until the EOS may be defined as t_{EOS}=min (t_{EOS.lvl}, t_{EOS.SNR}). Finally, a temporal smoothing filter may be applied to the estimated time such that the reported time until the EOS may be reported as t_{EOS}[n]=(1−0.03)×t_{EOS}[n−1]+0.03×t_{EOS}[n].

Example diagnostic session data may demonstrate characteristic behavior of the quality factors (QF), QF_{SNR}, and QFₗᵥₗ over the course of a measurement session. During an initial portion of a session following administration of the exogenous fluorescent agent, QF_{SNR} may gradually increase as the intrinsic fluorescence (IF) signal stabilizes and the renal decay dominated phase is approached. In some sessions, QF_{SNR} may exceed approximately 1.0, indicating high signal quality and sufficient stability for accurate tGFR reporting. In other sessions, QF_{SNR} may plateau below 1.0 but above a minimum acceptable threshold (e.g., approximately 0.5), at which point reporting may commence if the value has stabilized.

As the session progresses and agent concentration decreases, QFₗᵥₗ may gradually decline as the IF signal approaches baseline levels. QFₗᵥₗ may decrease slowly over time and may reach a limiting tolerance value (e.g., approximately 0.5), at which point an End of Session condition may be triggered. The combined QF, defined as the lower of QF_{SNR} and QFₗᵥₗ, may therefore initially rise during stabilization, remain relatively constant during steady renal decay dominated behavior, and then decline as signal level diminishes or noise increases.

Prediction of time remaining until End of Session may initially provide a relatively long estimate and may gradually decrease as IF signal levels decline. Changes in measured tGFR may cause corresponding increases or decreases in predicted remaining time, reflecting dynamic adaptation of the model to updated RDTC estimates. These example behaviors illustrate adaptive operation of the disclosed methods under varying physiological and signal conditions.

In some examples, a method to estimate the time to agent cleared based on tGFR, IF available, or eGFR is disclosed in which agent is sufficiently cleared in the current TGFR device iteration before a consecutive measurement can be undertaken. Accordingly, the time needed from End of Session to reach a level of residual agent that is low enough to not impede accuracy of subsequent GFR measurement with TGFR system may be determined through both a static and dynamic approach, as described herein.

Benefits of the embodiments described herein include, but are not limited to, accuracy of consecutive GFR measurements. If the monitoring should be prolonged over the duration of one measurement, it is inevitable that new age t is injected. It is desirable to keep the period between the end of transcutaneous monitoring and reinjection as short as possible in order to minimize the blind spot until a new tGFR reading is available. On the other hand, if reinjection happens too early, the signal from the remaining agent might skew the tGFR results. Further, accuracy of subsequent GFR measurement is ensured, in case first attempt to obtain tGFR after agent injection fails.

In some examples, during the EOS algorithm, whenever IF_{EOS} is calculated according to an equation shown below, an IF level may be set as an “agent cleared” IF level.

IF_{clr}=CLRMARGIN×IF_{EOS}, where CLRMARGIN is a constant, and IF_{clr} represents the IF signal below which the agent is expected to be sufficiently cleared (before the agent is injected, and IF_{clr} equal to the baseline IF value). Once the EOS has been declared, then IF_{clr} is updated according to IF_{clr}=F/RS×√(K×estimated RDTC)×IF_{ref}, where F, RS, and K are constants, IF_{ref} is the reference IF signal level calculated according to a flow-chart described in FIG. 13, and the RDTC is expressed in seconds, as described herein.

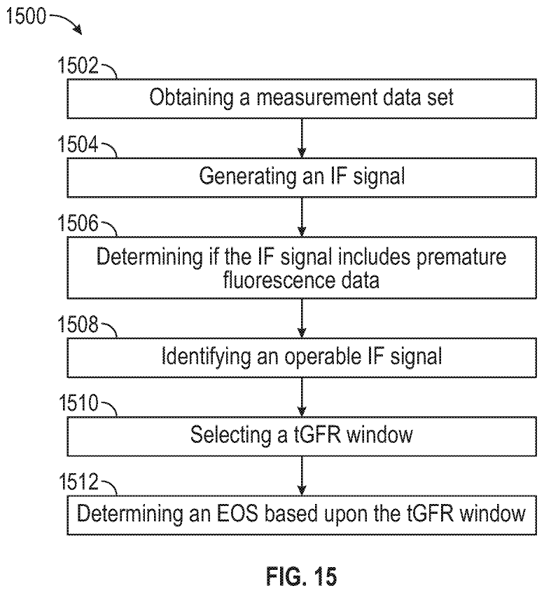

The reference IF signal is calculated from the IF signal levels recorded at 2 hours after the agent has been detected (IF@2) and at 3 hours after the agent has been detected (IF@3). The level chosen depends on the most recent estimated GFR values, as shown in the flow-chart shown in FIG. 15.

Accordingly, the tolerance for residual agent is allowed to increase as the RDTC increases (as the GFR decreases) according to the behavior of the tolerance model described herein. The GFR tolerance decreases with decreasing GFR and, therefore, amount of error that can be accepted due to a not fully complete returns to baseline. As a result, a longer wait for the residual agent level to fall farther at lower GFR values than at higher GFR values may be needed before allowing the start of a new session with a new agent injection. If this relaxation is not permitted in the agent decay tolerance, the ‘time to agent clearance’ would increase exponentially—to lengths of many days—at very small GFR (and long RDTC) values. An analysis of the standard error of our population shows that subjects with lower GFR, a higher ratio of GFR standard error over GFR (referenced herein as “error coefficient”) and can afford more inaccuracy due to a bit more residual agent. Further, a deviation caused by residual tracer of ¼th of the error coefficient can cause a residual agentlevel of approximately 4% for a subject with GFR=10, which may be reached after 2.7 days, which is still reasonable as these subjects are susceptible to longer monitoring periods already. For healthy subject, this corresponds with a residual agentlevel of approx. 1%.

In some examples, once the EOS algorithm, as described herein, has determined that there is insufficient agent to estimate the nGFR, this time until agent clearance algorithm estimates how long it will take for the agent to be fully cleared from the body based upon the last good IF signal value (IF_{good}) that is projected forward in time using the most recent good estimate of the RDTC, as described herein. The time until agent clearance is estimated as the time at which the projected signal is equivalent to the IF_{clr} signal level described above. Thus, the time until agent clearance t_{clearance} is calculated as t_{clearance}=−1×estimated RDTC×ln (IF_{clr}/IF_{good})−(t_{now}−t_{good}), where t_{now} is the current time, and t_{good} is the time at which the last good IF and RDTC values were recorded (the last time QF>RDTC_LOW_QUALITY_LIMIT). This calculation is performed once, immediately after End of Session has been declared. The time until agent clearance then simply counts down in seconds from this time.

FIG. 15 illustrates a flow chart 1500 corresponding to aspects of a method according to a flow-chart shown in FIG. 8. Although the method shown in FIG. 8 depicts a particular sequence of operations, the sequence may be altered without departing from the scope of the present disclosure.

According to some aspects, as depicted in FIG. 15, the method includes obtaining a measurement data set comprising a plurality of measurement entries that are obtained before and after administration of an exogenous fluorescent agent at block 1502. In aspects, the system and/or apparatus illustrated in FIG. 4 may obtain a measurement data set comprising a plurality of measurement entries that are obtained before and after administration of an exogenous fluorescent agent. In aspects, the system and/or apparatus can be used to collect data from the one or more sensor heads using the one or more light sources and one or more detectors. The collection of the data can be managed by the controller and/or processor. In at least one aspect, the controller and/or processor can store the data locally for processing. In another aspect, the data can be sent to a secure remote computer to perform the processing. The system and/or apparatus can be placed such that the one or more sensor heads are in proximity to or touching the patient from which the data is measured. The system and/or apparatus can be operational for a predetermined period of time prior to administration.

According to some aspects, the method includes generating an intrinsic fluorescence (IF) signal representing a detected fluorescence intensity emitted by the exogenous fluorescent agent from within a diffuse reflecting medium at block 1504. In aspects, the processor of the system illustrated in FIG. 4 may generate an IF signal representing a detected fluorescence intensity emitted by the exogenous fluorescent agent from within a diffuse reflecting medium.

According to some aspects, the method includes determining if the IF signal includes a portion of premature fluorescence data at block 1506. In aspects, the processor of the system illustrated in FIG. 4 may determine if the IF signal includes a portion of premature fluorescence data. In at least one aspect, the method at block 1506 includes determining a segment of decreasing IF signal. In other aspects, in determining a segment of decreasing IF signal, the method can identify a predetermined fitting window. The predetermined fitting window may have a length of time that is set at a predetermined length. The predetermined fitting window may adjust over time. In aspects, the predetermined fitting window has a fixed amount of time, but the window itself can move through the data.

According to some examples, the method includes at block 1508 identifying, upon determination of the portion of premature fluorescence data, an operable IF signal range by filtering the IF signal associated with premature fluorescence data. For example, the processor of the system illustrated in FIG. 4 may identify, upon determination of the portion of premature fluorescence data, an operable IF signal range by filtering the IF signal associated with premature fluorescence data.

According to some examples, the method includes selecting a transdermal Glomerular Filtration Rate (tGFR) window at block 1510, and determining an End of Session based upon the tGFR window at block 1512. As described herein, the tGFR window is selected using a signal-to-noise ratio (SNR) based quality factor (QF_{SNR}) or an IF signal level-based quality factor (QFₗᵥₗ). Further, the tGFR window is selected based upon a lower value of the QF SNR or QFₗᵥₗ. Further, the tGFR window is selected based upon a lower value of the QF SNR when a window size cannot be increased enough to compensate for noise. Similarly, the tGFR window is selected based upon a lower value of the QFₗᵥₗ using the IF signal level at 2 hours or 3 hours. The tGFR is above 75 mL/min/1.73 m2 when the IF signal is determined at 2 hour, and the tGFR is below 75 mL/min/1.73 m2 when the IF signal is determined at 3 hour. Additionally, the method includes adding a delay time before declaring the End of Session.

The methods provided herein offer several advantages to enhance the accuracy and reliability of tGFR measurements. The tGFR fitting window selection process uses the Quality Factor (QF) thresholds to ensure accurate reporting by avoiding reliance on noisy or insufficient data. For patients with stable tGFR, reporting is delayed until the data window is large enough to yield accurate results, while for patients experiencing changes in tGFR, shorter windows with higher QF allow faster detection of these changes. The QF_{SNR} method adapts window sizes based on noise levels, improving overall accuracy, and QFₗᵥₗ adjustments ensure longer windows are used as agent levels diminish, preventing baseline uncertainties from distorting results. Together, these methods dynamically optimize data collection for varying conditions, ensuring robust tGFR estimation.

Additionally, the methods incorporate advanced sensor displacement detection to maintain accuracy despite physical shifts. The methods differentiate between permanent sensor displacements, which terminate sessions to avoid inaccurate data, and temporary elastic changes caused by body movements, allowing recovery and continuation of accurate reporting. This prevents premature session termination and excludes corrupted data from calculations. End of Session detection further improves reliability by identifying when signal quality becomes insufficient, mitigating noise spikes and baseline drift to ensure sessions end only when necessary. Finally, the methods provide clinicians with a prediction of the remaining time for accurate tGFR reporting, offering critical guidance during measurement sessions.

What is claimed is:

1. A method of transdermal Glomerular Filtration Rate (tGFR) fitting window selection comprising:
obtaining a measurement data set comprising a plurality of measurement entries, wherein each measurement entry of the plurality of measurement entries is obtained before or after administration of an exogenous fluorescent agent;
calculating a renal decay time constant (RDTC), wherein the RDTC corresponds to a predetermined window that has a window length that is equal to or greater than a minimum window length and equal to or smaller than a maximum window length;
computing a quality factor (QF) corresponding to the predetermined window, wherein the QF includes a QF_{SNR} component and a QFₗᵥₗ component;
calculating a tGFR for the predetermined window, wherein the tGFR is calculated using data spanning the predetermined window; and
reporting the calculated tGFR.

2. The method of tGFR fitting window selection of claim 1, further comprising:
calculating a plurality of renal decay time constants (RDTCs), wherein each of the plurality of RDTCs corresponds to one of a plurality of predetermined windows, each of the plurality of predetermined windows having a window length that is equal to or greater than the minimum window length and equal to or smaller than the maximum window length;
computing a plurality of QFs, each of the plurality of QFs corresponding to a respective one of a plurality of predetermined window sizes, wherein each of the QFs include a QF_{SNR} component and a QFₗᵥₗ component;
calculating, when two or more of the plurality of QFs are within a predetermined deviation from one another, a mean RDTC from each of the plurality of predetermined windows corresponding to the two or more of the plurality of QFs;
selecting, when the plurality of QFs is more than the predetermined deviation from one another, one of the plurality of predetermined windows corresponding to a largest value of the plurality of QFs;
calculating a tGFR for the one of the predetermined windows corresponding to the largest value of the plurality of QFs or from the two or more predetermined windows corresponding to the two or more of the plurality of QFs, wherein the tGFR is calculated using data spanning the selected one of the predetermined window or from the two or more predetermined windows corresponding to the largest value of the plurality of QFs; and
reporting the calculated tGFR.

3. The method of tGFR fitting window selection of claim 2, wherein the QF_{SNR} component is calculated from a ratio of a measured window size to a limiting window size, wherein the measured window size is a window length used to generate the QF and the QFₗᵥₗ component is calculated from a ratio of an IF signal level to an End of a Session signal level.

4. The method of tGFR fitting window selection of claim 3, wherein computing the plurality of QFs comprises:
creating a base QF from a first QF;
calculating a QF for a next fitting window;
determining if the QF is greater than a predetermined minimum value and then determining if the QF is greater than the base QF;
updating, if a difference between the QF and the base QF is greater than a predetermined difference, the base QF with the value of the QF;
determining if additional fitting windows are needed based on an average of the QF and base QF being less than a predetermined value and repeating steps that follow until the average of the QF and base QF are larger than a predetermined minimum value:
calculating an additional QF for a next additional fitting window;
determining if the additional QF is greater than the predetermined minimum value and then determining if the additional QF is greater than the base QF; and
updating, if a difference between the additional QF and the base QF is greater than the predetermined difference, the base QF with the value of the additional QF;
reporting the base QF.

5. The method of tGFR fitting window selection of claim 4, wherein a short window is used to capture a changing value of tGFR and a long window is used to eliminate noise.

6. The method of tGFR fitting window selection of claim 5, wherein the creating of the base QF provides for a measurement in which the noise is below a desired threshold.

7. The method of tGFR fitting window selection of claim 3, further comprising:
determining a renal decay dominated phase when one or more of:
QF_{SNR} is at or above a predetermined high value; and
QF_{SNR} is above the predetermined minimum value and is not increasing, occurs.

8. The method of tGFR fitting window selection of claim 3, wherein the limiting window size is calculated from a noise model that determines if a signal to noise ratio falls within a normal distribution.

9. The method of tGFR fitting window selection of claim 2, further comprising:
generating an intrinsic fluorescence (IF) signal representing a detected fluorescence intensity emitted by the exogenous fluorescent agent from within a diffuse reflecting medium;
determining if the IF signal includes a portion of premature fluorescence data;
identifying, upon determination of the portion of premature fluorescence data, a range of operable IF signals by filtering the IF signal associated with premature fluorescence data;
determining that a peak has been reached in the IF signal; and
determining if the QF_{SNR} has a value that is above a minimum reporting threshold.

10. The method of tGFR fitting window selection of claim 9, wherein the minimum reporting threshold provides sufficient data quality to predict the tGFR.

11. The method of tGFR fitting window selection of claim 9, further comprising:
determining that the IF signal is in a renal decay dominated phase based upon passing of the peak and the QF_{SNR} having a value above the minimum reporting threshold.

12. A method of detecting sensor displacement for a system operable to measure Glomerular Filtration Rate (GFR) using an exogenous fluorescent agent, the method comprising:
generating an intrinsic fluorescence (IF) signal representing a detected fluorescence intensity emitted by the exogenous fluorescent agent from within a diffuse reflecting medium based on measurements from one or more sensors;
detecting a change in the IF signal corresponding to potential displacement of the one or more sensors relative to a patient, wherein the potential displacement corresponds with a perturbation in at least one raw optical signal;
determining a peak has been reached in the IF signal;
continuing to calculate the IF signal for one or more predetermined time periods if the peak has been reached and comparing IF signal after each of the one or more predetermined time periods relative to a predicted IF signal based on a calculation from a selected time prior to the potential displacement;
continuing with measurements when the calculated IF signal is less than or equal to a predetermined deviation from an expected IF value; and
declaring an End of Session if the calculated IF signal is more than the predetermined deviation from the expected IF value.

13. The method of claim 12, further comprising:
continuing to calculate the IF signal for a shortened predetermined period of time if the peak has not yet been reached and comparing IF signal after the shortened predetermined period of time relative to a predicted IF signal based on a calculation from the selected time prior to the potential displacement; and
declaring an End of Session if the calculated IF signal is more than the predetermined deviation from the expected IF value.

14. The method of claim 12, further comprising:
providing a measurement data set comprising a plurality of measurement entries comprising at least one diffuse reflectance signal at an excitation wavelength of the exogenous fluorescent agent (DRₑₓ), a diffuse reflectance signal at an emission wavelength of the exogenous fluorescent agent (DRₑₘ), and a fluorescence emission (Flr) signal;
estimating a first signal median within a first window;
estimating a second signal median within a second window;
calculating a difference between the first signal median and the second signal median for each of DRₑₓ, DRₑₘ, and Flr;
normalizing the difference to a corresponding signal level;
calculating a first absolute difference by taking an absolute value of a difference in the DRₑₓ minus a difference in DRₑₘ;
calculating a second absolute difference by taking an absolute value of a difference in the Flr minus a difference in DRₑₓ; and
declaring the potential displacement if both the first absolute difference and the second absolute difference are greater than a nominal threshold.

15. The method of claim 14, further comprising:
creating a reference IF value from IF data at a predetermined time prior to the potential sensor displacement;
projecting the IF signal over a predetermined time interval using a last known RDTC value;
determining a difference between the projected IF signal and an actual IF measurement at the potential sensor displacement;
declaring the potential sensor displacement to be insignificant if the actual IF measurements are within a predetermined percentage of the actual IF measurements;
declaring the potential sensor displacement to be significant if the actual IF measurements are outside a predetermined percentage of the actual IF measurements.

16. The method of claim 15, wherein the projecting the IF signal uses:
$${IF}_{p r j} ( t ) = {IF}_{r e f} {e}^{- t / RDTC} .$$

17. The method of claim 15, wherein a time window of between ten and thirty minutes is used to determine whether the potential sensor displacement is temporary or permanent.

18. The method of claim 17, further comprising:
for each window of ten minutes spanning a maximum time of thirty minutes, calculating:
$$Δ IF = ( IF - IFref ) / IFref ; and$$
$$Δ IFnom = ( IFprj - IFref ) / IFref ;$$
and
declaring that the sensor is currently displaced if |ΔIF−ΔIFnom|>(4.5×Last CV).

19. The method of claim 12, further comprising:
excluding from the IF signal, upon detection of a potential sensor displacement over a displacement period, a portion of the IF signal calculated over the displacement period.

20. A method of detecting an End of a Glomerular Filtration Rate (GFR) Session using an exogenous fluorescent agent, the method comprising:
computing a quality factor (QF) corresponding to a predetermined window of measurement data set comprising a plurality of measurement entries obtained before or after administration of an exogenous fluorescent agent, wherein the QF includes a QF_{SNR} component and a QFₗᵥₗ component;
calculating a transdermal Glomerular Filtration Rate (tGFR) for the predetermined window, wherein the tGFR is calculated using data spanning the predetermined window;
assigning a lower of QF_{SNR} or QFₗᵥₗ as an end determining QF; and
declaring an End of Session once the end determining QF falls below a limiting tolerance level.

21. The method of claim 20, wherein the limiting tolerance level is a level below which noise is no longer insignificant.

22. The method of claim 20, further comprising:
obtaining a measurement data set comprising a plurality of measurement entries that are obtained before and/or after administration of an exogenous fluorescent agent;
generating an intrinsic fluorescence (IF) signal representing a detected fluorescence intensity emitted by the exogenous fluorescent agent from within a diffuse reflecting medium;
setting a first recorded IF reference signal at a first predetermined time after the exogenous fluorescent agent has been detected;
setting a second recorded IF reference signal at a second predetermined time after the exogenous fluorescent agent has been detected, wherein the second predetermined time is greater than the first predetermined time;
calculating invalid decay ratio based upon a smaller ratio of either a current IF signal to the first recorded IF reference signal or the current IF signal to the second recorded IF signal;
declaring a possible End of Session when the invalid decay ratio is less than a predetermined End of Session value.

23. The method of claim 22, further comprising:
determining if the IF signal includes a portion of premature fluorescence data;
identifying, upon determination of the portion of premature fluorescence data, a range of operable IF signals by filtering the IF signal associated with the portion of the premature fluorescence data;
wherein the first recorded IF reference signal and the second recorded IF reference signal occur after a peak in the IF signal.

24. The method of claim 22, further comprising, if the End of Session is determined based upon the QFₗᵥₗ component of the QF:
resetting the End of Session if the QF rises above the limiting tolerance level during a time delay.

25. The method of claim 24, wherein the time delay can be a predetermined time delay or a calculated time delay.

26. The method of claim 22, further comprising, if the End of Session is determined based upon the QF_{SNR} component of the QF:
calculating a time delay based on one of:
if the QF_{SNR} is below a predetermined limiting tolerance level, setting a first time delay;
if the QF_{SNR} is above the predetermined limiting tolerance level, setting a second time delay; and
resetting the End of Session if the QF rises above the limiting tolerance level during the first time delay or the second time delay.

27. A method of predicting a time until an End of a Glomerular Filtration Rate (GFR) Session using an exogenous fluorescent agent, the method comprising:
obtaining a measurement data set comprising a plurality of measurement entries that are obtained before and after administration of an exogenous fluorescent agent;
generating an intrinsic fluorescence (IF) signal representing a detected fluorescence intensity emitted by the exogenous fluorescent agent from within a diffuse reflecting medium;
saving a last known good signal level as IF_{good};
saving a last known good RDTC value as RDTC_{good};
calculating the time until the End of the GFR Session based upon when an End of Session time will occur such that an End of Session will be declared, wherein the End of Session time is a function of a current signal level and a current signal to noise ratio.

28. The method of claim 27, further comprising:
calculating
$${t}_{E O S , l v l} = - 1 \times RDT {C}_{g o o d} \times ln \frac{{IF}_{E O S}}{{IF}_{g o o d}} + \frac{1}{2} {W}_{min}$$
$$calculating$$
$${t}_{E O S , S N R} = - 1 \times RDT {C}_{g o o d} \times ln ( \frac{{k}_{c x}}{{IF}_{g o o d}} ) + {W}_{a d j}$$
$$wherein :$$
$${k}_{c x} = {( \frac{{CV}_{lim}}{σ} {k}_{c p} )}^{\frac{1}{f P O W E R - 1}} ;$$
$${k}_{c p} = {IF}_{good}^{fPOWER} , wherein {f}_{POWER} is an empirically - derived constant ;$$
$$σ = C {V}_{g o o d} \times {IF}_{g o o d} ; and$$
$${W}_{a d j} = min ( {fMARGIN}_{FRAC} \times ( {W}_{g o o d} + {fMARGIN}_{MIN} ) , {fMARGIN}_{FRAC} \times {W}_{max} ) ;$$
wherein the End of Session is a minimum of t_{EOS,lvl} and t_{EOS,SNR}, and
wherein W_{good} is a last known good fitting window size, Wₘᵢₙ is the minimum window size, W_{MAX} is a maximum fitting window size, IF_{EOS} is the IF signal at the End of Session, CVₗᵢₘ is a function of RDTC and fitting window size, CV_{good} is a coefficient of variation of the last known good fitting window size, fMARGIN_FRAC is 0.6, and fMARGIN_MIN is 30.0.

29. The method of claim 27, further comprising applying a smoothing filter to an estimated time, so that time until the End of the GFR Session is calculated as:
reported t_{EOS}[n]=(1−0.03)×t_{EOS}[n−1]+0.03×t_{EOS}[n].

30. A method of determining a waiting period length before an exogenous fluorescent agent can be re-injected, the method comprising:
determining a baseline level of auto-fluorescence prior to injection of the exogenous fluorescent agent;
generating an intrinsic fluorescent (IF) signal;
calculating a transdermal Glomerular Filtration Rate (tGFR);
estimating, based upon the tGFR, a residual agent concentration; and
determining a calculated time, after which the residual agent concentration does not cause significant bias on the tGFR, wherein the calculated time is used as when a second injection of the exogenous fluorescent agent can be made.

31. The method of claim 30, further comprising determining a wait time from the tGFR.

32. The method of claim 30, further comprising determining a wait time from an estimated GFR (eGFR).

33. The method of claim 30, wherein the calculated time is determined by minimizing the period of time from a last tGFR from a first injection of the exogenous fluorescent agent to a second injection of the exogenous fluorescent agent.
